SEQUENCE LISTING (4 sequences)

SEQ ID NO: 1 - protein, 525 aa, Homo sapiens
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 2 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 3 - protein, 312 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQEDQSSTGFRLKTSSTRKYK

SEQ ID NO: 4 - protein, 343 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGFLMRTNT

(19) United States
(12) Patent Application Publication
(10) Pub. No.: US 2017/0362632 A1
(43) Pub. Date: Dec. 21, 2017

(54) ENZYMATIC ACTIVITY ASSAYS FOR I2S

(71) Applicant: Shire Human Genetic Therapies, Inc., Lexington, MA (US)

(72) Inventors: Peter BERNHARDT, Lexington, MA (US); Claire TIEN, Lexington, MA (US); Elisha FIELDING, Lexington, MA (US); Vijay CHHAJLANI, Lexington, MA (US)

(21) Appl. No.: 15/537,224

(22) PCT Filed: Dec. 17, 2015
(86) PCT No.: PCT/US15/66263

§ 371 Date: Jun. 16, 2017

Related U.S. Application Data
(60) Provisional application No. 62/093,826, filed on Dec. 18, 2014.

(51) Int. Cl. C12Q 1/34

(52) U.S. Cl. CPC C12Q 1/34; G01N 2333/916

(57) ABSTRACT

In certain embodiments of the present invention, kinetic parameters of I2S enzyme are determined. In some instances, a sample including I2S enzyme is incubated under defined conditions, with a series of determined amounts of I2S substrate including a detectable label. Following incubation, the reaction mixture can be analyzed, e.g., by a method including chromatography. A detection unit can be used to measure the presence of the detectable label. Data can be analyzed to determine kinetic parameters.

### CROSS REFERENCE TO RELATED APPLICATIONS

This application claims the benefit of U.S. Provisional Application No. 62/093,826, filed Dec. 18, 2014, the contents of which are hereby incorporated herein in their entirety.

### BACKGROUND

Iduronate-2-sulfatase (I2S), is a member of the sulfatase family and is capable of catalyzing the removal of a sulfate group from compounds such as dermatan sulfate and heparan sulfate.

Deficiency of I2S can result in clinical phenotypes. Specifically, the absence of or deficiency in I2S enzyme in patients with Hunter Syndrome can lead to progressive accumulation of glycosaminoglycans (GAGs), e.g., dermatan sulfate or heparan sulfate, in the lysosomes of a variety of cell types, potentially leading to cellular engorgement, organomegaly, tissue destruction, and organ system dysfunction. Generally, physical manifestations of Hunter Syndrome include both somatic and neuronal symptoms. In some cases of Hunter Syndrome, central nervous system involvement leads to developmental delays and nervous system problems. GAG accumulation in the peripheral tissue can lead to a distinctive coarseness in the facial features of a patient and is responsible for the prominent forehead, flattened bridge and enlarged tongue. Accumulation of GAG can adversely affect the organ systems of the body. Manifesting initially as a thickening of the wall of the heart, lungs and airways, and abnormal enlargement of the liver, spleen and kidneys, these profound changes can ultimately lead to widespread catastrophic organ failure. Hunter Syndrome is typically severe, progressive, and life-limiting.

An important treatment for Hunter Syndrome is enzyme replacement therapy (ERT). For example, ERT for Hunter Syndrome can include administering replacement I2S enzyme to patients with Hunter Syndrome. ELAPRASE®, manufactured by Shire plc, is a purified recombinant form of I2S approved by the FDA as an enzyme replacement therapy for the treatment of Hunter Syndrome.

### SUMMARY

The present invention provides, among other things, improved methods for assessing potency of I2S to facilitate enzyme replacement therapy. In particular, the present invention provides enzyme activity assays for I2S and recombinant forms thereof using a physiologically relevant substrate, e.g., a substrate that is representative of one or more physiological substrates that accumulate in patients suffering from Hunter Syndrome. Thus, the present invention permits more clinically relevant assessment of recombinant I2S for enzyme replacement therapy.

At least one aspect of the present invention includes a method of determining the potency of iduronate-2-sulfatase (I2S), including the steps of contacting a sample including iduronate-2-sulfatase (I2S) with a substrate containing a terminal iduronate-2-sulfate under conditions that permit the I2S-catalyzed desulfation of the substrate, which I2S-catalyzed desulfation of the substrate is associated with a detectable signal, and detecting the detectable signal, thereby determining one or more kinetic parameters or the specific activity of the I2S, such that the one or more kinetic parameters or the specific activity can be indicative of the potency and/or active properties of I2S. In particular embodiments, the substrate is defined by a structure of formula I:

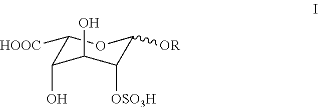

or a suitable salt thereof wherein R is hydrogen, a carbohydrate domain optionally substituted with a detectable moiety, an oxygen protecting group, a detectable moiety, or an optionally substituted group selected from the group consisting of C₁₋₁₂ aliphatic, phenyl, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, 5- to 6-membered heteroaryl having 1-4 heteroatoms selected from oxygen, nitrogen, or sulfur, 7- to 10-membered saturated or partially unsaturated bicyclic carbocyclyl, 7- to 10-membered saturated or partially unsaturated bicyclic heterocyclyl having 1-4 heteroatoms selected from oxygen, nitrogen, or sulfur, 7- to 10-membered bicyclic heteroaryl having 1-4 heteroatoms selected from oxygen, nitrogen, or sulfur, or 8- to 10-membered bicyclic aryl; and [symbol] indicates an α- or β-anomer, or a mixture thereof. In certain embodiments, the detectable group is a fluorescent group. In particular embodiments, R is 4-methylumbelliferyl (4MU). In some embodiments, the detectable group is detectable via chemiluminescence or ultraviolet/visible absorbance spectroscopy.

In certain embodiments, the substrate can be 4-methylumbelliferyl-α-L-idopyranosiduronic acid 2-sulfate (IdoA2S-4MU):

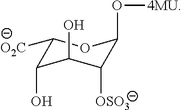

In certain embodiments, the I2S-catalyzed desulfation generates an I2S product, and the product can be IdoA-4MU:

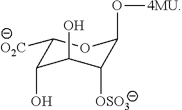

In any of the above embodiments, the step of detecting the detectable signal can include performing chromatography. For instance, in some embodiments, the chromatography is selected from the group consisting of ion chromatography, high-performance liquid chromatography (HPLC), ultra performance liquid chromatography, and a combination thereof. In some embodiments, the step of analyzing the product formation includes performing ultra-performance or high-performance liquid chromatography coupled to fluorescence detection. The chromatography can include at least one column selected from a BEH amide, HILIC, RP, or CSH column.

In any of the above embodiments, the step of detecting the detectable signal can include determining the amount of the product as compared to a control. In particular embodiments, the control is a pre-determined amount of the product or a product standard curve.

In any of the above embodiments, the step of detecting the detectable signal can include determining the rate of product formation.

In any of the above embodiments, the one or more kinetic parameters can be selected from the group consisting of Vₘₐₓ, Kₘ, k_{cat}, specific activity. and combination thereof. In certain embodiments, the one or more kinetic parameters are determined by fitting data obtained from detecting the detectable signal to the Michaelis-Menten model or other kinetic models suitable to determine kinetic parameters.

In any of the above embodiments, the sample can be a drug substance, a drug product, or a stability sample of drug substance and drug product.

In any of the above embodiments, the conditions that permit I2S-catalyzed desulfation of the substrate can include incubation at about 37° C. for about 20 minutes, a buffer pH of approximately 4-5, and in the presence of BSA.

In any of the above embodiments, the conditions that permit the I2S-catalyzed desulfation of the substrate can include between 0 and 0.4 mg/mL BSA.

In any of the above embodiments, the method can further include a step of quenching the desulfation by addition of acetonitrile, or another organic solvent, water-miscible or not miscible, or by using heat denaturation.

In various embodiments, the present invention includes a method of determining the potency of iduronate-2-sulfatase (I2S), which method includes steps of: contacting a sample comprising iduronate-2-sulfatase (I2S) with a substrate containing a terminal iduronate-2-sulfate under conditions that permit the I2S-catalyzed desulfation of the substrate, which I2S-catalyzed desulfation of the substrate is associated with a detectable signal, and detecting the detectable signal, thereby determining one or more kinetic parameters or the specific activity of the I2S, such that the one or more kinetic parameters or the specific activity is indicative of the potency and active properties of I2S. In some embodiments, the substrate is defined by a structure of formula I or a suitable salt thereof where R is hydrogen, a carbohydrate domain optionally substituted with a detectable moiety, an oxygen protecting group, a detectable moiety, or an optionally substituted group selected from the group consisting of C₁₋₁₂ aliphatic, phenyl, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, 5- to 6-membered heteroaryl having 1-4 heteroatoms selected from oxygen, nitrogen, or sulfur, 7- to 10-membered saturated or partially unsaturated bicyclic carbocyclyl, 7- to 10-membered saturated or partially unsaturated bicyclic heterocyclyl having 1-4 heteroatoms selected from oxygen, nitrogen, or sulfur, 7- to 10-membered bicyclic heteroaryl having 1-4 heteroatoms selected from oxygen, nitrogen, or sulfur, or 8- to 10-membered bicyclic aryl; and indicates an α- or β-anomer, or a mixture thereof. In some embodiments, such a method further comprises contacting the sample with iduronidase (IDUA) under conditions that permit generation of detectable 4-MU, e.g., where the contacting of the sample with IDUA is after the I2S-catalyzed desulfation of the substrate. In certain embodiments, the conditions that permit generation of detectable 4-MU include adding a high pH quench reagent.

### Definitions

In order for the present invention to be more readily understood, certain terms are first defined below. Additional definitions for the following terms and other terms are set forth throughout the specification.

Batch: As used herein, the term “batch” refers to a completed manufacturing run, in which a product, finished good or component is produced. In some embodiments, a batch comprises multiple “lots”. As used herein, the term “lot” refers to a part or fraction of the total completed product produced during the manufacture of a commercial batch. In some embodiments, a batch consists of a single lot. In some embodiments, a batch consists of a plurality of lots. In some embodiments, a batch is partitioned into individual lots based on sample size, FDA requirements and/or shipping conditions. In some embodiments, a batch is partitioned into lots based on specific factions produced during manufacture of the batch.

Biologically active: As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. Biological activity can also be determined by in vitro assays (for example, in vitro enzymatic assays). In particular embodiments, where a protein or polypeptide is biologically active, a portion of that protein or polypeptide that shares at least one biological activity of the protein or polypeptide is typically referred to as a “biologically active” portion. In some embodiments, a protein is produced and/or purified from a cell culture system, which displays biologically activity when administered to a subject.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control. In some embodiments, the control may be a “reference control”, which is a sample used for comparison with a test sample, to look for differences or for the purposes of characterization.

Concentration: As used herein, the term “concentration” refers to a measure indicative of amount of substance in a volume. Typically, concentration is measured by a numerical value with physical units of mass*volume⁻¹, such as molar and millimolar.

Enzyme: As used herein, the term “enzyme” refers to any protein capable of producing changes in a biological substance by catalytic action.

Enzyme activity: As used herein, the term “enzyme activity”, “enzymatic activity” or grammatical equivalent, refers to the general catalytic properties of an enzyme.

Enzyme assays: As used herein, the term “enzyme assays”, “enzymatic assays”, “enzymatic activity assays”, or grammatical equivalent, refers to procedures for measuring the amounts or activities of enzyme in a sample.

Enzyme reaction: As used herein, the term “enzyme reaction” refers to a chemical process in which an enzyme catalyzes conversion of one or more molecules into different molecules. Molecules at the beginning of the process are called substrates. Molecules at the end of the process are called products.

Enzyme replacement therapy (ERT): As used herein, the term “enzyme replacement therapy (ERT)” refers to any therapeutic strategy that corrects an enzyme deficiency by providing the missing enzyme. In some embodiments, the missing enzyme is provided by intrathecal administration. In some embodiments, the missing enzyme is provided by infusing into bloodstream. Once administered, enzyme is taken up by cells and transported to the lysosome, where the enzyme acts to eliminate material that has accumulated in the lysosomes due to the enzyme deficiency. Typically, for lysosomal enzyme replacement therapy to be effective, the therapeutic enzyme is delivered to lysosomes in the appropriate cells in target tissues where the storage defect is manifest.

Kinetic model: As used herein, the term “kinetic model” refers to any quantitative description of enzyme reaction rate. Typically, a kinetic model constitutes an equation to fit kinetic experimental data and/or derive a set of parameters that define an enzymatic reaction. For example, a Michaelis-Menten kinetic model is a common model of a single-substrate reaction. A kinetic model may require, benefit from, or optionally include, in various instances, particular assumptions or requisites for application. Such assumptions or requisites are known in the art with respect to particular kinetic models, e.g., the Michaelis-Menten model.

Kinetic parameter: As used herein, the term “kinetic parameter” means any measure relating to the activity of an enzyme in a particular enzymatic reaction with a particular substrate. As used herein, kinetic parameters include any parameters indicative of reaction rate (e.g., Vₘₐₓ and Kₘ, etc.) and specific activity. Under the Michaelis-Menten model, the substrate concentration (denoted as [S]) must be greater than the enzyme concentration (denoted as [E]), and initial rates must be determined for each [S]. Vₘₐₓ represents the maximum rate achieved by the system, at saturating substrate concentrations. Typically, enzyme-catalyzed reactions are saturable. Their rate of catalysis does not always show a linear response to increasing substrate. If the initial rate of the reaction is measured over a range of substrate concentrations (denoted as [S]), the reaction rate (v) generally increases as [S] increases. However, as [S] gets higher, the enzyme becomes saturated with substrate and the rate reaches V, the enzyme's maximum rate. Kₘ also known as the Michaelis constant, is the substrate concentration at which the reaction rate is half of Vₘₐₓ. The parameter k_{cat} is equal to Vₘₐₓ/[E].

Specific activity is typically defined as the amount of substrate the enzyme converts (reactions catalyzed), per mg protein in the enzyme preparation, per unit of time. Specific activity can be used as to calculate or estimate activity recovery following purification.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Various other sequence alignment programs are available and can be used to determine sequence identity such as, for example, Clustal.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Potency: As used herein, the term “potency” refers to the specific ability or capacity of a product, as indicated by appropriate tests (e.g., an enzymatic kinetic assay described herein), to effect a desired therapeutic result. In some instances, potency is quantitatively indicated by appropriate tests (e.g., an enzymatic kinetic assay described herein). In some instance, potency is qualitatively indicated by appropriate tests (e.g., an enzymatic kinetic assay described herein). For instance, the potency of a product may be indicated by various kinetic parameters including, without limitation, Vₘₐₓ, Kₘ, k_{cat}, specific activity, or any combination thereof, measured by an enzymatic assay described herein. Thus, in some embodiments, an enzymatic kinetic assay described herein may be used as potency test.

Replacement enzyme: As used herein, the term “replacement enzyme” refers to any enzyme that can act to replace at least in part the deficient or missing enzyme in a disease to be treated. In some embodiments, the term “replacement enzyme” refers to any enzyme that can act to replace at least in part the deficient or missing lysosomal enzyme in a lysosomal storage disease to be treated. In some embodiments, a replacement enzyme is capable of reducing accumulated materials in mammalian lysosomes or that can rescue or ameliorate one or more lysosomal storage disease symptoms. Replacement enzymes suitable for the invention include both wild-type or modified lysosomal enzymes and can be produced using recombinant and synthetic methods or purified from nature sources. A replacement enzyme can be a recombinant, synthetic, gene-activated or natural enzyme.

Sample: As used herein, the term “sample” means a small part of something intended to show the quality, nature or quantity of the whole thing. The term sample encompasses any sample obtained from any source. For example, a sample containing an enzyme of interest may be obtained from an enzyme production system, enzyme purification process, formulated drug substance, or a biological source. In some embodiments, the term “sample” encompasses a composition (e.g., an assay mixture, reaction, reaction intermediate, processed form thereof, or purified form thereof, together with any admixed reagents) including all or a portion of a starting sample or all or a portion of enzyme present in a starting sample.

Standard Curve: As used herein, the term “standard curve” refers to a type of graph used as a quantitative research tool. Typically, multiple samples with known properties are measured and graphed, which then allows the same properties to be determined for unknown samples by interpolation on the graph. The samples with known properties are the standards, and the graph is the standard curve.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena. In some embodiments, the phrase “substantially pure” of “substantially purified”, refers to a protein (native or recombinant) which is substantially free of contaminating endogenous materials, such as, e.g., other proteins, lipids, carbohydrates, nucleic acids and other biological materials with which it is naturally associated. For example, a substantially pure molecule can be at least about 60%, by dry weight, preferably about 70%, 80%, 90%, 95% or 99% of the molecule of interest.

The abbreviations used herein have their conventional meaning within the chemical and biological arts. The chemical structures and formulae set forth herein are constructed according to the standard rules of chemical valency known in the chemical arts.

### BRIEF DESCRIPTION OF THE DRAWINGS

FIG. 1 is an exemplary diagram showing I2S-catalyzed desulfation of the physiologically relevant substrate IdoA2S-4MU to form the product IdoA-4MU.

FIG. 2 is an exemplary diagram of an exemplary 96-well dilution plate layout. Each standard curve column contains a serial dilutions series of the product IdoA-4MU. Each well of the sample columns includes 80 μL of 0.02 ng/μL I2S sample. Only one set of IdoA-4MU standards is necessary for the entire experiment.

FIG. 3 is an exemplary diagram of an exemplary 96-well assay plate. Each well of columns 1-3 contain 40 μL of product in particular concentrations. The enzymatic reaction occurs in the remaining wells, which include 20 μL of experimental or control I2S sample and 20 μL of IdoA2S-4MU substrate.

FIG. 4 is an exemplary graph exemplifying chromatographic data acquired according to a method of the present invention.

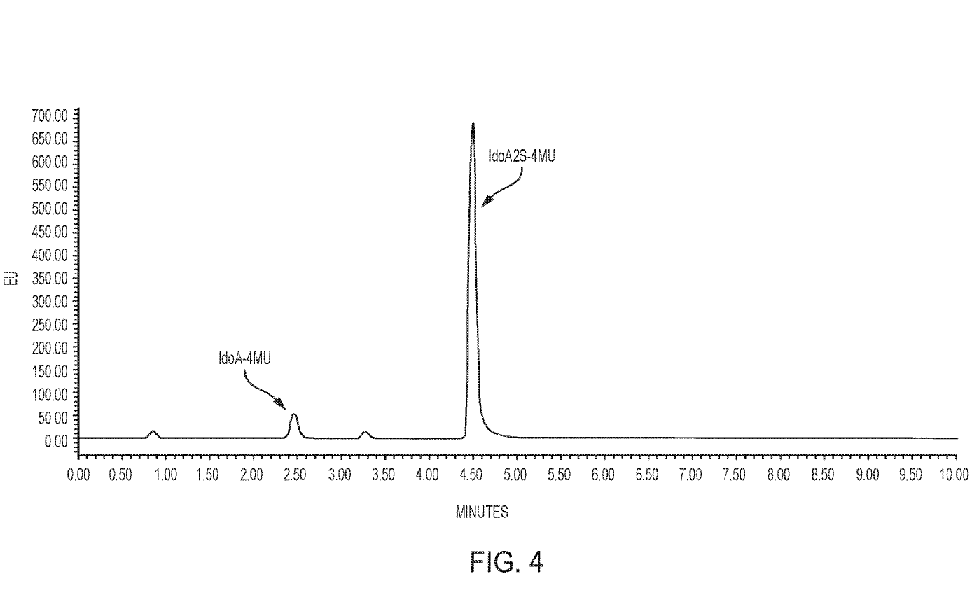

FIG. 5 is a schematic of a reaction. The schematic shows a reaction having two steps. In the first step, 4-methylumbelliferyl α-L-idopyranosiduronic acid 2-sulfate (IdoA2S-MU) is hydrolyzed to sulfate and 4-methylumbelliferyl α-L-idopyranosiduronic acid (IdoA-MU). The first step is catalyzed by idursulfase or iduronate-2-sulfatase (I2S). In the second step, 4-methylumbelliferyl α-L-idopyranosiduronic acid (IdoA-MU) is hydrolyzed to idopyranosiduronic acid (IdoA) and 4-methylumbelliferone (4-MU). The second step is catalyzed by iduronidase (IDUA). The schematic further illustrates as part of the second step a high pH quench generating an anionic version of 4-MU that is measurable by fluorescence.

### DETAILED DESCRIPTION

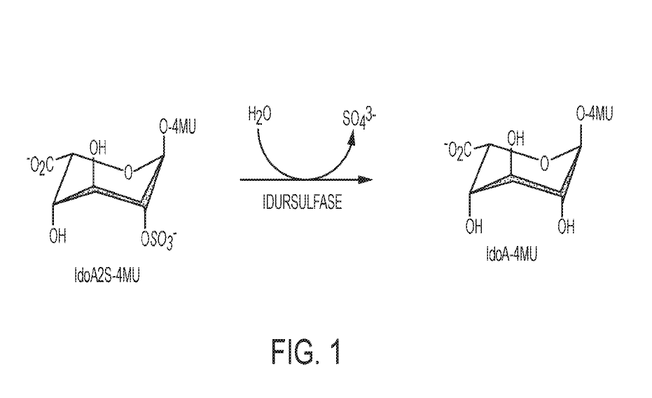

The present invention provides, among other things, methods and compositions for determining enzyme kinetic parameters (e.g., Vmax, Km, and specific activity, etc.) or specific activity indicative of clinically relevant potency of idursulfase (I2S) enzyme using a physiologically relevant substrate, e.g., a substrate that interacts with I2S enzyme in a manner representative of one or more substrates that accumulate in patients suffering from Hunter Syndrome or of a complex mixture of heterogeneous polymers that typically accumulate in patients suffering from Hunter Syndrome. For example, I2S enzyme catalyzes the desulfation of terminal iduronate 2-sulfate (IdoA2S) under physiological conditions to generate iduronate (IdoA) and sulfate (FIG. 1). The present invention provides, among other things, a discontinuous assay for the determination of kinetic parameters or specific activity relating to I2S.

In certain examples of the present invention, the product generated when I2S enzyme acts on a physiologically relevant I2S substrate is detected. An I2S substrate may include a detectable group, such as a fluorescent detectable group. One example of a physiologically relevant substrate is IdoA2S-4MU (FIG. 1). Data collected from such an assay can be analyzed to determine kinetic parameters or specific activity relating to I2S enzyme.

In certain examples of the present invention, a product generated when I2S enzyme acts on a physiologically relevant I2S substrate is further modified or treated prior to a detection step or step in which product generation is measured. For instance, in some instances, a first step in which I2S enzyme acts on a physiologically relevant I2S substrate is followed by a second step that further modifies a product generated by the action of the I2S enzyme on a physiologically relevant I2S substrate. For example, the second step may result in modification to such a generated product in a manner that enables one or more particular detection steps, analysis steps, methods of detection, or methods of analysis. In a particular example, a product generated by the action of the I2S enzyme on a physiologically relevant I2S substrate is not detectable by fluorescence, is not detectable by fluorescence at a particular wavelength, is insufficiently detectable by fluorescence, and/or is insufficiently detectable by fluorescence at a particular wavelength, at least with respect to one or more techniques of analysis or detection. In certain embodiments a product generated by the action of the I2S enzyme on a physiologically relevant I2S substrate is rendered detectable or more detectable by fluorescence or fluorescence at a particular wavelength, at least with respect to one or more techniques of analysis or detection by a subsequent or second step.

In various embodiments of the present invention, a subsequent or second step may include a high pH quench. A high pH quench may include contacting an enzyme, sample, or product produced by reaction of a starting substrate with a high pH quench reagent. A high pH quench reagent may be any acceptable solution having a high pH, e.g., a pH greater than 7, greater than 7.5, greater than 8, greater than 8.5, greater than 9, greater than 9.5, greater than 10, greater than 10.5, greater than 11, greater than 11.5, greater than 12, greater than 12.5, or greater than 13. In various embodiments, a high pH quench reagent may be any acceptable solution having a high pH, e.g., a pH between 7 and 14, a pH between 8 and 13, a pH between 9 and 12, a pH between 10 and 11, or any of various possible ranges existing between pH 7 and pH 14. In specific instances, a high pH quench reagent may have a pH of about 10.7. A variety of agents or combinations of agents that produce a high pH solution are known in the art. A variety of agents or combinations of agents that produce a high pH solution sufficient to mediate a high pH quench are known in the art. A variety of agents or combinations of agents that produce a high pH solution such that the solution is capable of mediating an increasing in anionic character of a molecule upon contact with the molecule are known in the art. In certain particular instances, a high pH quench may modify a molecule or product, e.g., 4-MU or a 4-MU precursor (e.g., the product of IDUA-catalyzed hydrolysis of IdoA-Mu), in a manner than renders it detectable by fluorescence. In certain instances, a high pH quench reagent may include sodium carbonate, e.g., more than 0.01 M sodium carbonate, more than 0.05 M sodium carbonate, more than 0.1 M sodium carbonate, more than 0.2 M sodium carbonate, more than 0.3 M sodium carbonate, more than 0.4 M sodium carbonate, more than 0.5 M sodium carbonate, more than 0.6 M sodium carbonate, more than 0.7 M sodium carbonate, more than 0.8 M sodium carbonate, more than 0.9 M sodium carbonate, or more than 1 M sodium carbonate. In various instances, a high pH quench reagent may include a surfactant, e.g., Triton X-100. In various instances, a high pH quench reagent can include, e.g., 0.001% or more surfactant such as Triton X-100, 0.005% or more surfactant such as Triton X-100, 0.01% or more surfactant such as Triton X-100, 0.02% or more surfactant such as Triton X-100, 0.03% or more surfactant such as Triton X-100, 0.04% or more surfactant such as Triton X-100, 0.05% or more surfactant such as Triton X-100, or 0.1% or more surfactant such as Triton X-100.

In some embodiments, a subsequent or second step by which a product generated by the action of the I2S enzyme on a physiologically relevant I2S substrate is rendered detectable or more detectable by fluorescence or fluorescence at a particular wavelength, at least with respect to one or more techniques, includes reacting such a product with a further enzyme. In some specific instances the further enzyme is iduronidase (IDUA). In some such instances, IDUA catalyzes hydrolysis of such a product in a manner that renders the downstream product detectable or more detectable by fluorescence or fluorescence at a particular wavelength, at least with respect to one or more techniques of analysis or detection.

In some instances, a subsequent or second step as described above includes, incorporates, is concurrent with, is preceded by, or is followed by a high-pH step that contributes to, causes, or completes, a reaction that renders a product generated by the action of the I2S enzyme on a physiologically relevant I2S substrate detectable or more detectable by fluorescence or fluorescence at a particular wavelength, at least with respect to one or more techniques.

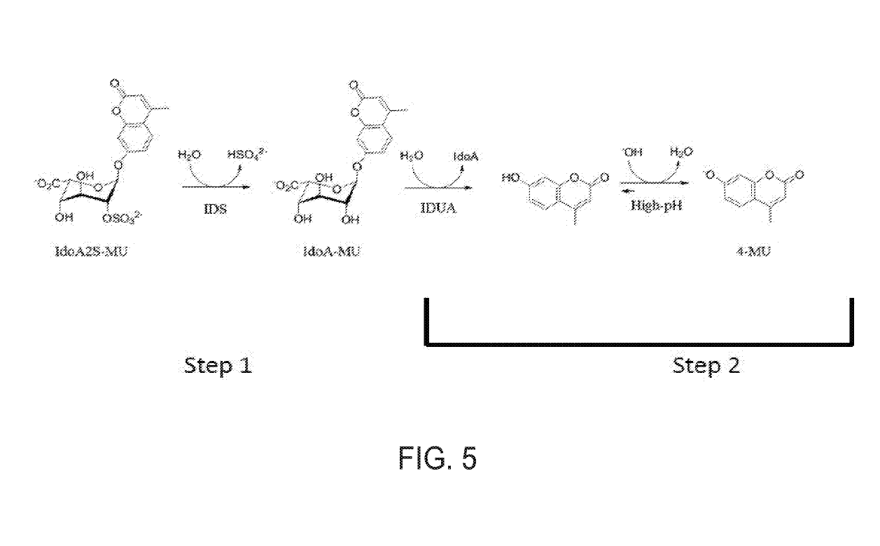

In a specific instance exemplified herein, a multi-step reaction is provided for determination of I2S kinetic parameters or specific activity. In certain embodiments, such a multi-step reaction includes one or more steps in accordance with the schematic of FIG. 5, at least in that it includes a first step in which 4-methylumbelliferyl α-L-idopyranosiduronic acid 2-sulfate (IdoA2S-MU) is hydrolyzed to sulfate and 4-methylumbelliferyl α-L-idopyranosiduronic acid (IdoA-MU), which first step is catalyzed by idursulfase or iduronate-2-sulfatase (I2S).

In a specific instance exemplified herein, a multi-step reaction is provided for determination of I2S kinetic parameters or specific activity. In certain embodiments, such a multi-step reaction includes one or more steps in accordance with the schematic of FIG. 5, at least in that it includes a second step in which 4-methylumbelliferyl α-L-idopyranosiduronic acid (IdoA-MU) is hydrolyzed to idopyranosiduronic acid (IdoA) and 4-methylumbelliferone (4-MU). The second step is catalyzed by iduronidase (IDUA).

In a specific instance exemplified herein, a multi-step reaction is provided for determination of I2S kinetic parameters or specific activity. In certain embodiments, such a multi-step reaction includes one or more steps in accordance with the schematic of FIG. 5, at least in that it includes a second step in which a high pH quench generates an anionic version of 4-MU that is measurable by fluorescence. In various embodiments, such high pH quench occurs conccurently with, before, or after hydrolysis of 4-methylumbelliferyl α-L-idopyranosiduronic acid (IdoA-MU) to idopyranosiduronic acid (IdoA) and 4-methylumbelliferone (4-MU).

Accordingly, certain specific embodiments providing methods and compositions for determining enzyme kinetic parameters or specific activity indicative of clinically relevant potency of idursulfase (I2S) enzyme include a multi-step reaction for determination of I2S kinetic parameters or specific activity as outlined in the schematic of FIG. 5. In such embodiments, an assay includes two steps. In a first step, 4-methylumbelliferyl α-L-idopyranosiduronic acid 2-sulfate (IdoA2S-MU) is hydrolyzed to sulfate and 4-methylumbelliferyl α-L-idopyranosiduronic acid (IdoA-MU). The first step is catalyzed by idursulfase or iduronate-2-sulfatase (I2S). In a second step, 4-methylumbelliferyl α-L-idopyranosiduronic acid (IdoA-MU) is hydrolyzed to idopyranosiduronic acid (IdoA) and 4-methylumbelliferone (4-MU). The second step is catalyzed by iduronidase (IDUA). The schematic further illustrates as part of the second step a high pH quench generating an anionic version of 4-MU that is measurable by fluorescence.

Thus, present invention is particularly useful to measure the enzyme activity and kinetic properties of I2S enzyme in a drug substance, drug product, or stability sample for enzyme replacement therapy.

Various aspects of the invention are described in detail in the following sections. The use of sections is not meant to limit the invention. Each section can apply to any aspect of the invention. In this application, the use of “or” means “and/or” unless stated otherwise.

### I2S Enzyme

The present invention may be applied to any I2S enzyme, including any recombinant or naturally-occurring I2S enzyme. The human I2S gene (IDS) is typically located at Xq28 and can encode a 525-amino acid glycoprotein with a molecular weight of approximately 76 kilodaltons. The enzyme can contain eight asparagine-linked glycosylation sites capable of being occupied by complex oligosaccharide structures. The enzyme activity of I2S can be dependent on the post-translational modification of a specific cysteine to formylglycine. I2S can be capable of cleaving the terminal 2-O-sulfate moieties from the glycosaminoglycans (GAG) dermatan sulfate and heparan sulfate.

As used herein, the terms “I2S enzyme,” “I2S protein,” and grammatical equivalents, are used interchangeably. A single sample of I2S enzyme may include multiple forms of the enzyme.

The present invention is applicable to naturally-occurring I2S enzymes (e.g., wild-type or mutant forms) or I2S enzymes produced through in vivo or in vitro gene or protein recombination, engineering, de novo synthesis, combinations thereof, or other techniques of molecular biology. In some embodiments, a I2S enzyme suitable for the present invention is any protein or a portion of a protein (e.g., comprising at least 30, 35, 40, 45, 50, 60, 70, 80, 90, 100, 200, 300, 400, 450, 500, 525, 550, or more, or all amino acids) having at least 70% homology or identity (e.g., 70, 71, 72, 73, 74, 75, 80, 85, 90, 95, or 100% homology or identity) to a naturally-occurring or recombinant I2S enzyme, including, but not limited to, those recombinant I2S proteins described herein.

I2S enzymes of the present invention may be derived from a variety of sources or present in a variety of contexts. In some instances, an I2S enzyme of the present invention is present in a cell by which it is encoded or in an organism including a cell by which it is encoded. In other instances, an I2S enzyme is present in a sample taken from an organism including a cell that encodes it. In some cases, the I2S enzyme is purified from a cell, organism, or sample. In some cases, I2S enzyme is produced by cells of a cell culture. Cells encoding an I2S enzyme may be cultured under laboratory conditions such that I2S enzyme is produced by the cells, and a sample of purified or unpurified I2S can be taken or derived therefrom. In other instances, I2S is synthesized using cell-free methods of synthesis. In particular circumstances, an I2S enzyme is present in a pharmaceutical composition. In certain instances, an I2S enzyme is administered to a cell or organism. In such instances, an I2S enzyme sample can be taken from a cell or organism that does not encode it.

Typically, the human I2S protein is produced as a precursor form. The precursor form of human I2S contains a signal peptide (amino acid residues 1-25 of the full length precursor), a pro-peptide (amino acid residues 26-33 of the full length precursor), and a chain (residues 34-550 of the full length precursor) that may be further processed into the 42 kDa chain (residues 34-455 of the full length precursor) and the 14 kDa chain (residues 446-550 of the full length precursor). Typically, the precursor form is also referred to as full-length precursor or full-length I2S protein, which contains 550 amino acids. The amino acid sequences of the mature form (SEQ ID NO: 1) having the signal peptide removed and full-length precursor (SEQ ID NO: 2) of a typical wild-type or naturally-occurring human I2S protein are shown in Table 1. The signal peptide is underlined. In addition, the amino acid sequences of human I2S protein isoform a and b precursor are also provided in Table 1, SEQ ID NO: 3 and 4, respectively.

TABLE 1
Human Iduronate-2-sulfatase

| | |
|---|---|
| Mature Form | SETQANSTTDALNVLLIIVDDLRPSLGCYGDKLVRSPN |
| | IDQLASHSLLFQNAFAQQAVCAPSRVSFLTGRRPDTTR |
| | LYDFNSYWRVHAGNFSTIPQYFKENGYVTMSVGKVFHP |
| | GISSNHTDDSPYSWSFPPYHPSSEKYENTKTCRGPDGE |
| | LHANLLCPVDVLDVPEGTLPDKQSTEQAIQLLEKMKTS |
| | ASPFFLAVGYHKPHIPFRYPKEFQKLYPLENITLAPDP |
| | EVPDGLPPVAYNPWMDIRQREDVQALNISVPYGPIPVD |
| | FQRKIRQSYFASVSYLDTQVGRLLSALDDLQLANSTII |
| | AFTSDHGWALGEHGEWAKYSNFDVATHVPLIFYVPGRT |
| | ASLPEAGEKLFPYLDPFDSASQLMEPGRQSMDLVELVS |
| | LFPTLAGLAGLQVPPRCPVPSFHVELCREGKNLLKHFR |
| | FRDLEEDPYLPGNPRELIAYSQYPRPSDIPQWNSDKPS |
| | LKDIKIMGYSIRTIDYRYTVWVGFNPDEFLANFSDIHA |
| | GELYFVDSDPLQDHNMYNDSQGGDLFQLLMP |
| | (SEQ ID NO: 1) |
| Full-Length | MPPPRTGRGLLWLGLVLSSVCVALGSETQANSTTDALN |
| Precursor | VLLIIVDDLRPSLGCYGDKLVRSPNIDQLASHSLLFQN |
| (Isoform a) | AFAQQAVCAPSRVSFLTGRRPDTTRLYDFNSYWRVHAG |
| | NFSTIPQYFKENGYVTMSVGKVFHPGISSNHTDDSPYS |
| | WSFPPYHPSSEKYENTKTCRGPDGELHANLLCPVDVLD |
| | VPEGTLPDKQSTEQAIQLLEKMKTSASPFFLAVGYHKP |
| | HIPFRYPKEFQKLYPLENITLAPDPEVPDGLPPVAYNP |
| | WMDIRQREDVQALNISVPYGPIPVDFQRKIRQSYFASV |
| | SYLDTQVGRLLSALDDLQLANSTIIAFTSDHGWALGEH |
| | GEWAKYSNFDVATHVPLIFYVPGRTASLPEAGEKLFPY |
| | LDPFDSASQLMEPGRQSMDLVELVSLFPTLAGLAGLQV |
| | PPRCPVPSFHVELCREGKNLLKHFRFRDLEEDPYLPGN |
| | PRELIAYSQYPRPSDIPQWNSDKPSLKDIKIMGYSIRT |
| | IDYRYTVWVGFNPDEFLANFSDIHAGELYFVDSDPLQD |
| | HNMYNDSQGGDLFQLLMP (SEQ ID NO: 2) |
| Isoform b | MPPPRTGRGLLWLGLVLSSVCVALGSETQANSTTDALN |
| Precursor | VLLIIVDDLRPSLGCYGDKLVRSPNIDQLASHSLLFQN |
| | AFAQQAVCAPSRVSFLTGRRPDTTRLYDFNSYWRVHAG |
| | NFSTIPQYFKENGYVTMSVGKVFHPGISSNHTDDSPYS |
| | WSFPPYHPSSEKYENTKTCRGPDGELHANLLCPVDVLD |
| | VPEGTLPDKQSTEQAIQLLEKMKTSASPFFLAVGYHKP |
| | HIPFRYPKEFQKLYPLENITLAPDPEVPDGLPPVAYNP |
| | WMDIRQREDVQALNISVPYGPIPVDFQEDQSSTGFRLK |
| | TSSTRKYK (SEQ ID NO: 3) |
| Isoform c | MPPPRTGRGLLWLGLVLSSVCVALGSETQANSTTDALN |
| Precursor | VLLIIVDDLRPSLGCYGDKLVRSPNIDQLASHSLLFQN |
| | AFAQQAVCAPSRVSFLTGRRPDTTRLYDFNSYWRVHAG |
| | NFSTIPQYFKENGYVTMSVGKVFHPGISSNHTDDSPYS |
| | WSFPPYHPSSEKYENTKTCRGPDGELHANLLCPVDVLD |
| | VPEGTLPDKQSTEQAIQLLEKMKTSASPFFLAVGYHKP |
| | HIPFRYPKEFQKLYPLENITLAPDPEVPDGLPPVAYNP |
| | WMDIRQREDVQALNISVPYGPIPVDFQRKIRQSYFASV |
| | SYLDTQVGRLLSALDDLQLANSTIIAFTSDHGFLMRTN |
| | T (SEQ ID NO: 4) |

In some embodiments, a recombinant I2S protein is mature human I2S protein (SEQ ID NO:1). As disclosed herein, SEQ ID NO:1 represents the canonical amino acid sequence for the human I2S protein. In some embodiments, the I2S protein may be a splice isoform and/or variant of SEQ ID NO:1, resulting from transcription at an alternative start site within the 5′ UTR of the I2S gene. In some embodiments, a recombinant I2S protein may be a homologue or an analogue of mature human I2S protein. For example, a homologue or an analogue of mature human I2S protein may be a modified mature human I2S protein containing one or more amino acid substitutions, deletions, and/or insertions as compared to a wild-type or naturally-occurring I2S protein (e.g., SEQ ID NO:1), while retaining substantial I2S protein activity. Thus, in some embodiments, a recombinant I2S protein is substantially homologous to mature human I2S protein (SEQ ID NO:1). In some embodiments, a recombinant I2S protein has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 1. In some embodiments, a recombinant I2S protein is substantially identical to mature human I2S protein (SEQ ID NO:1). In some embodiments, a recombinant I2S protein has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO: 1. In some embodiments, a recombinant I2S protein contains a fragment or a portion of mature human I2S protein.

Alternatively, a recombinant I2S protein is full-length I2S protein. In some embodiments, a recombinant I2S protein may be a homologue or an analogue of full-length human I2S protein. For example, a homologue or an analogue of full-length human I2S protein may be a modified full-length human I2S protein containing one or more amino acid substitutions, deletions, and/or insertions as compared to a wild-type or naturally-occurring full-length I2S protein (e.g., SEQ ID NO:2), while retaining substantial I2S protein activity. Thus, In some embodiments, a recombinant I2S protein is substantially homologous to full-length human I2S protein (SEQ ID NO:2). For example, a recombinant I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:2. In some embodiments, a recombinant I2S protein is substantially identical to SEQ ID NO:2. For example, a recombinant I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:2. In some embodiments, a recombinant I2S protein contains a fragment or a portion of full-length human I2S protein. As used herein, a full-length I2S protein typically contains signal peptide sequence.

In some embodiments, a recombinant I2S protein is human I2S isoform a protein. In some embodiments, a recombinant I2S protein may be a homologue or an analogue of human I2S isoform a protein. For example, a homologue or an analogue of human I2S isoform a protein may be a modified human I2S isoform a protein containing one or more amino acid substitutions, deletions, and/or insertions as compared to a wild-type or naturally-occurring human I2S isoform a protein (e.g., SEQ ID NO:3), while retaining substantial I2S protein activity. Thus, in some embodiments, a recombinant I2S protein is substantially homologous to human I2S isoform a protein (SEQ ID NO:3). For example, a recombinant I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:3. In some embodiments, a recombinant I2S protein is substantially identical to SEQ ID NO:3. For example, a recombinant I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:3. In some embodiments, a recombinant I2S protein contains a fragment or a portion of human I2S isoform a protein. As used herein, a human I2S isoform a protein typically contains a signal peptide sequence.

In some embodiments, a recombinant I2S protein is human I2S isoform b protein. In some embodiments, a recombinant I2S protein may be a homologue or an analogue of human I2S isoform b protein. For example, a homologue or an analogue of human I2S isoform b protein may be a modified human I2S isoform b protein containing one or more amino acid substitutions, deletions, and/or insertions as compared to a wild-type or naturally-occurring human I2S isoform b protein (e.g., SEQ ID NO:4), while retaining substantial I2S protein activity. Thus, in some embodiments, a recombinant I2S protein is substantially homologous to human I2S isoform b protein (SEQ ID NO:4). For example, a recombinant I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:4. In some embodiments, a recombinant I2S protein is substantially identical to SEQ ID NO:4. For example, a recombinant I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:4. In some embodiments, a recombinant I2S protein contains a fragment or a portion of human I2S isoform b protein. As used herein, a human I2S isoform b protein typically contains a signal peptide sequence.

Homologues or analogues of human I2S proteins can be prepared according to methods for altering polypeptide sequence known to one of ordinary skill in the art such as are found in references that compile such methods. In some embodiments, conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In some embodiments, a “conservative amino acid substitution” refers to an amino acid substitution that does not alter the relative charge or size characteristics of the protein in which the amino acid substitution is made.

In some embodiments, recombinant I2S proteins may contain a moiety that binds to a receptor on the surface of target cells to facilitate cellular uptake and/or lysosomal targeting. For example, such a receptor may be the cation-independent mannose-6-phosphate receptor (CI-MPR) which binds the mannose-6-phosphate (M6P) residues. In addition, the CI-MPR also binds other proteins including IGF-II. In some embodiments, a recombinant I2S protein contains M6P residues on the surface of the protein. In particular, a recombinant I2S protein may contain bis-phosphorylated oligosaccharides which have higher binding affinity to the CI-MPR. In some embodiments, a suitable enzyme contains up to about an average of about at least 20% bis-phosphorylated oligosaccharides per enzyme. In other embodiments, a suitable enzyme may contain about 10%, 15%, 18%, 20%, 25%, 30%, 35%, 40%, 45%, 50%, 55%, 60% bis-phosphorylated oligosaccharides per enzyme.

In some embodiments, recombinant I2S enzymes may be fused to a lysosomal targeting moiety that is capable of binding to a receptor on the surface of target cells. A suitable lysosomal targeting moiety can be IGF-I, IGF-II, RAP, p97, and variants, homologues or fragments thereof (e.g., including those peptide having a sequence at least 70%, 75%, 80%, 85%, 90%, or 95% identical to a wild-type mature human IGF-I, IGF-II, RAP, p97 peptide sequence). The lysosomal targeting moiety may be conjugated or fused to an I2S protein or enzyme at the N-terminus, C-terminus or internally.

The present inventions includes any I2S enzymes provided herein, within the definition of I2S enzyme provided herein, or that may be derived from natural or laboratory-induced mutation of a naturally-occurring I2S or other I2S enzyme.

### I2S Enzyme Samples

Samples of I2S enzyme may be derived from a variety of sources. Samples of I2S can include, without limitation, drug substance, drug product, samples derived from cell lines, cell lines, stored samples, or in-process samples. A suitable sample for the present invention may contain I2S enzyme in any form (e.g., isolated or not, purified or unpurified). In particular embodiments, a suitable sample for the present invention is a sample containing a purified I2S enzyme for enzyme replacement therapy, also referred to as replacement I2S enzyme. Such a sample may be a drug substance, drug product, or a stability sample. Purified replacement I2S enzyme may be a recombinant, synthetic, gene-activated or natural enzyme.

In some embodiments, a suitable sample for the present invention contains recombinant I2S enzyme. As used herein, the term recombinant I2S enzyme refers to any I2S enzyme produced using a recombinant technology. Suitable expression systems for recombinant technology include, for example, egg, baculovirus, plant, yeast, or mammalian cells. In some embodiments, a recombinant I2S is produced by cells engineered to express I2S. Typically, cells encoding an I2S enzyme may be cultured under standard cell culture conditions such that I2S enzyme is produced by the cells.

In some embodiments, I2S enzymes are produced in cells, e.g., mammalian cells. Non-limiting examples of mammalian cells that may be used in accordance with the present invention include BALB/c mouse myeloma line (NSO/I, ECACC No: 85110503); human retinoblasts (PER.C6, CruCell, Leiden, The Netherlands); monkey kidney CV1 line transformed by SV40 (COS-7, ATCC CRL 1651); human embryonic kidney line (HEK293 or 293 cells subcloned for growth in suspension culture, Graham et al., J. Gen Virol., 36:59, 1977); human fibrosarcoma cell line (e.g., HT1080); baby hamster kidney cells (BHK21, ATCC CCL 10); Chinese hamster ovary cells +/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

In some embodiments, recombinant I2S enzymes produced from human cells (e.g., HT1080) are purified. In some embodiments, recombinant I2S enzymes produced from CHO cells are purified.

Typically, cells that are engineered to express I2S may comprise a transgene that encodes an I2S protein described herein. It should be appreciated that the nucleic acids encoding I2S may contain regulatory sequences, gene control sequences, promoters, non-coding sequences and/or other appropriate sequences for expressing the I2S. Typically, the coding region is operably linked with one or more of these nucleic acid components. An I2S sample may be purified according to any of a variety of methods known in the art.

In some instances a sample is taken from an organism and contains a naturally-occurring I2S enzyme. Such a sample may be derived, for example, from a tissue sample (e.g., a tissue biopsy, e.g. an organ biopsy), from drawn blood, from bodily fluids, or by other means known in the art. An I2S enzyme sample may be derived for a mammal, such as a mouse, rat, guinea pig, dog, cat, horse, pig, non-human primate, or human. Samples may be used with or without further processing. In some instances a sample may be sterilized, homogenized, diluted, disassociated, or processed to isolate particular cell types or cellular components, such as lysosomes. Methods thereof are well known to those of skill in the art.

In some instances, the I2S enzyme present in an I2S enzyme sample is activated. I2S enzyme may be activated by any of a variety of methods known in the art. Typically, a recombinant I2S enzyme is activated by the post-translational modification of a conserved cysteine (corresponding to amino acid 59 of mature human I2S) to formylglycine, also known as 2-amino-3-oxopropionic acid, or oxo-alanine. Such post-translational modification can be carried out by an enzyme known as Formylglycine Generating Enzyme (FGE). Thus, in some embodiments, recombinant I2S enzymes are produced in cells that also express FGE protein. In particular embodiments, recombinant I2S enzymes are produced in cells that have increased or enhanced expression of FGE protein. For example, cells may be engineered to over-express FGE in combination with recombinant I2S to facilitate the production of I2S preparations having high levels of active enzyme. In some embodiments, over-expression of FGE is achieved by expression (e.g., over-expression) of an exogenous FGE using standard recombinant technology. In some embodiments, over-expression of FGE is achieved by activated or enhanced expression of an endogenous FGE by, for example, activating or enhancing the promoter of the endogenous FGE gene. In some cases, the nucleic acid encoding recombinant I2S and the nucleic acid encoding a recombinant FGE protein are linked by a nucleic acid (e.g., a spacer sequence) having a sequence corresponding to an internal ribosomal entry site.

Any FGE having ability to convert cysteine to formylglycine may be used in the present invention. Exemplary nucleic acid and amino acid sequences for FGE proteins are disclosed in US 2004-0229250, the entire contents relating to such sequences and the sequences themselves are incorporated herein by reference in their entireties. It should be appreciated that the nucleic acids encoding recombinant FGE may comprise regulatory sequences, gene control sequences, promoters, non-coding sequences and/or other appropriate sequences for expressing the FGE. Typically, the coding region is operably linked with one or more of these nucleic acid components.

I2S enzyme samples may be intermediates in a process of therapeutic production, including without limitation purified 125 enzyme not yet processed into a therapeutic form.

### Physiologically Relevant Substrates of I2S Enzyme

As used herein, the term “physiologically relevant substrate” refers to any substrate that 125 enzyme is able to catalyze the desulfation of, the substance including a reactive moiety (i.e., iduronate-2-sulfate) that is representative of a moiety present in a complex mixture of heterogeneous polymers that typically accumulates in patients suffering from I2S enzyme deficiency, such as Hunter Syndrome. In some embodiments, a physiologically relevant substrate suitable for the present invention is IdoA2S-4MU. I2S is known to cleave the terminal 2-O-sulfate moieties from the glycosaminoglycans (GAGs) dermatan sulfate and heparan sulfate. Thus, GAGs typically accumulate abnormally in various tissues of patients suffering from Hunter Syndrome, where I2S enzyme is absent or nonfunctional.

Thus, in some embodiments, a physiological substrate suitable for the present invention can be a compound that includes a GAG moiety, such as a dermatan sulfate or heparan sulfate moiety.

In some embodiments, I2S substrates of the present invention include molecules defined by a structure of formula I:

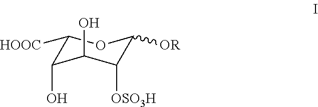

or a suitable salt thereof wherein R is hydrogen, a carbohydrate domain optionally substituted with a detectable moiety, an oxygen protecting group, a detectable moiety, or an optionally substituted group selected from the group consisting of C₁₋₁₂ aliphatic, phenyl, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, 5- to 6-membered heteroaryl having 1-4 heteroatoms selected from oxygen, nitrogen, or sulfur, 7- to 10-membered saturated or partially unsaturated bicyclic carbocyclyl, 7- to 10-membered saturated or partially unsaturated bicyclic heterocyclyl having 1-4 heteroatoms selected from oxygen, nitrogen, or sulfur, 7- to 10-membered bicyclic heteroaryl having 1-4 heteroatoms selected from oxygen, nitrogen, or sulfur, or 8- to 10-membered bicyclic aryl; and [symbol] indicates an α- or β-anomer, or a mixture thereof.

In some embodiments, R is a detectable moiety. In some embodiments, R is a fluorescent group. In some embodiments, R is a detectable moiety that is activated by desulfation of a compound of Formula I. In some embodiments, R is a detectable moiety that is activated by contacting a compound of Formula I with idursulfase. In some embodiments, R is −4MU.

In some embodiments, R is or comprises a carbohydrate domain optionally substituted with a detectable moiety. In some embodiments, R is or comprises a monosaccharide optionally substituted with a detectable moiety. In some embodiments, R is or comprises a disaccharide optionally substituted with a detectable moiety. In some embodiments, R is or comprises an oligosaccharide optionally substituted with a detectable moiety. In some embodiments, R is or comprises a glycosaminoglycan optionally substituted with a detectable moiety. In some embodiments, R is a carbohydrate domain having the structure:

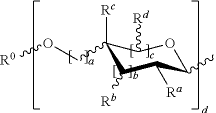

wherein:
each occurrence of a, b, and c is independently 0, 1, or 2;
d is an integer from 1-5, wherein each d bracketed structure may be the same or different; with the proviso that the d bracketed structure represents a furanose or pyranose moiety, and the sum of b and c is 1 or 2;
R⁰ is hydrogen, an oxygen protecting group or an optionally substituted moiety selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR^{x}, N(R′)₂, NHCOR′, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, or sulfur; 4-7-membered heterocyclyl having 1-2 heteroatoms independently selected from the group consisting of nitrogen, oxygen, and sulfur;
R′ is R′ or an oxygen protecting group; and
each occurrence of R′ is independently hydrogen, or an optionally substituted group selected from acyl, —S(O)₃, arylalkyl, 6-10-membered aryl, C₁₋₁₂ aliphatic, or C₁₋₁₂ heteroaliphatic having 1-2 heteroatoms independently selected from the group consisting of nitrogen, oxygen, and sulfur; or:
two R′ on the same nitrogen atom are taken with the nitrogen to form a 4-7-membered heterocyclic ring having 1-2 heteroatoms independently selected from the group consisting of nitrogen, oxygen, and sulfur;
wherein the carbohydrate domain is optionally substituted with a detectable moiety.

In some embodiments, the [symbol] bond represents α-anomeric stereochemistry. In some embodiments, the [symbol] bond represents β-anomeric stereochemistry. In some embodiments, provided I2S substrates comprise a mixture of α- and β-isomers at the position of [symbol] depicted in Formula I.

In some embodiments, I2S substrates of the present invention include molecules defined by a structure of formula I-a:

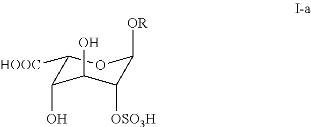

or a suitable salt thereof, wherein R is as defined above and described in classes and subclasses herein.

In some embodiments, I2S substrates of the present invention include molecules defined by a structure of formula I-b:

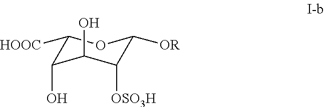

or a suitable salt thereof, wherein R is as defined above and described in classes and subclasses herein.

In some embodiments, I2S substrates of the present invention are salts of formula I. In some embodiments, such substrates are depicted without counterions. In some embodiments, I2S substrates of the present invention are defined by a structure of formula II:

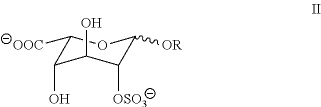

wherein R is as defined above and described in classes and subclasses herein.

In some embodiments, I2S substrates of the present invention are defined by a structure of formula II-a:

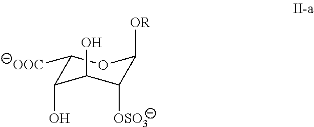

wherein R is as defined above and described in classes and subclasses herein.

In some embodiments, I2S substrates of the present invention are defined by a structure of formula II-b:

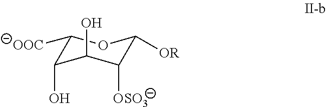

wherein R is as defined above and described in classes and subclasses herein.

In certain embodiments of the present invention, a physiologically relevant substrate can be detectably labeled with a detectable group to enable the qualitative or quantitative assessment of kinetic parameters or specific activity of I2S enzyme that acts upon the substrate. “Detectable moiety” is used interchangeably with the terms “detectable group”, “label”, and “reporter” and relates to any moiety capable of being detected, e.g., primary labels and secondary labels. In certain embodiments, a detectable moiety is a fluorescent label. The terms “fluorescent label”, “fluorescent dye”, and “fluorophore” as used herein refer to moieties that absorb light energy at a defined excitation wavelength and emit light energy at a different wavelength.

An exemplary detectable group of the present invention is 4-MU. Detectable groups further include hydroxy- and amino-substituted coumarins and fluorescent 7-hydroxy coumarin compounds with substitutions in the 4 position having a length greater than one carbon atom, which may be related to 4-MU. Some examples of such 7-hydroxy coumarins include phosphate, ester and ether derivatives of 7-hydroxy-4-methylcoumarin (β-methylumbelliferone), typified by 4-methylumbelliferyl phosphate (MUP), 7-hydroxy-4-methylcoumarin, 6,8-Difluoro-7-hydroxy-4-methylcoumarin (DiFMU), and the 7-hydroxycoumarin fluorophorethe phosphate ester of 6,8-difluoro-7-hydroxy-4-methylcoumarin (DiFMUP). In some embodiments, a detectable moiety is selected from the group consisting of 4-methylumbelliferyl-β-D-glucuronide (MUG), 4-methylumbelliferyl-β-D-glucoside (MBGL), 4-methylumbelliferyl-β-d-galactoside (MBGA), 4-methylumbelliferyl-alpha-d galactoside (MAGA), and 4-methylumbelliferyl-alpha-D-glucoside (MAGL). Other non-limiting examples of a detectable moiety include nitrophenol substrates such as o-nitro-phenol-β-D galactopyranoside (ONPG), p-nitro-phenol-N-acetyl-β-D-glucosaminide (NAG), p-nitrophenol-alpha-D-fucopyranoside (AFU), O-nitrophenl-alpha-D-glucopyranoside (AGLU), and PO4-(alkaline phosphatase), as well as naphthylamide substrates such as arginine β-naphthylamide (ARG), proline-β-naphthylamide (PRO), pyrrolidonyl-β-naphthylamide (PYR), Na-Benzoyl-DL-arginine-β-naphtylamide-“trypsin” (TRY), N-Glutaryl-Gly-Gly-Phe-β-naphthylamide-“chymotrypsin” (CHY), and Leucyl glycine β naphthylamide (LGY). Still other detectable groups that may be used include BODIPY, paranitrophenyl, Resorufin, naphthyl labels, 1,9-dimethylmethylene blue (DMMB), toluidine blue, Alcian blue, and related labels.

Additional examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-5, BODIPY dyes (BODIPY FL, BODIPY R6G, BODIPY TMR, BODIPY TR, BODIPY 493/503, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X, 5(6)-Carboxyfluorescein, 2,7-Dichlorofluorescein, N,N-Bis(2,4,6-trimethylphenyl)-3,4:9,10-perylenebis(dicarboximide, HPTS, Ethyl Eosin, DY-490XL MegaStokes, DY-485XL MegaStokes, Adirondack Green 520, ATTO 465, ATTO 488, ATTO 495, YOYO-1,5-FAM, BCECF, dichlorofluorescein, rhodamine 110, rhodamine 123, YO-PRO-1, SYTOX Green, Sodium Green, SYBR Green I, Alexa Fluor 500, FITC, Fluo-3, Fluo-4, fluoro-emerald, YoYo-1 ssDNA, YoYo-1 dsDNA, YoYo-1, SYTO RNASelect, Diversa Green-FP, Dragon Green, EvaGreen, Surf Green EX, Spectrum Green, NeuroTrace 500525, NBD-X, MitoTracker Green FM, LysoTracker Green DND-26, CBQCA, PA-GFP (post-activation), WEGFP (post-activation), FlASH-CCXXCC, Azami Green monomeric, Azami Green, green fluorescent protein (GFP), EGFP (Campbell Tsien 2003), EGFP (Patterson 2001), Kaede Green, 7-Benzylamino-4-Nitrobenz-2-Oxa-1,3-Diazole, Bexl, Doxorubicin, Lumio Green, and SuperGlo GFP.

A presence of a detectable moiety can be measured using methods for quantifying (in absolute, approximate or relative terms) the detectable moiety in a system under study. In some embodiments, such methods are well known to one of ordinary skill in the art and include any methods that quantify a reporter moiety (e.g., a label, a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, quantum dot(s), a novel functional group, a group that covalently or noncovalently interacts with other molecules, a photocaged moiety, an actinic radiation excitable moiety, a ligand, a photoisomerizable moiety, biotin, a biotin analog (e.g., biotin sulfoxide), a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, and any combination of the above).

Primary labels, such as radioisotopes (e.g., tritium, ³²P, ³³P, ³⁵S, ¹⁴C, ¹²³I, ¹²⁴I, ¹²⁵I, or ¹³¹I), mass-tags including, but not limited to, stable isotopes (e.g., ¹³C, ²H, ¹⁷O, ¹⁸O, ¹⁵N, ¹⁹F, and ¹²I), positron emitting isotopes (e.g., ¹¹C, ¹⁸F, ¹³N, ¹²⁴I, and ¹⁵O), and fluorescent labels are signal generating reporter groups which can be detected without further modifications. Detectable moieties may be analyzed by methods including, but not limited to fluorescence, positron emission tomography, SPECT medical imaging, chemiluminescence, electron-spin resonance, ultraviolet/visible absorbance spectroscopy, mass spectrometry, nuclear magnetic resonance, magnetic resonance, flow cytometry, autoradiography, scintillation counting, phosphoimaging, and electrochemical methods. In particular embodiments, a detectable moiety is detectable via chemiluminescence or ultraviolet/visible absorbance spectroscopy.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,819, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags. Stable isotopes (e.g., ¹³C, ²H, ¹⁷O, ¹⁸O, and ¹⁵N) may also be used as mass-tags.

The term “chemiluminescent group,” as used herein, refers to a group which emits light as a result of a chemical reaction without the addition of heat. By way of example, luminol (5-amino-2,3-dihydro-1,4-phthalazinedione) reacts with oxidants like hydrogen peroxide (H₂O₂) in the presence of a base and a metal catalyst to produce an excited state product (3-aminophthalate, 3-APA).

The term “chromophore,” as used herein, refers to a molecule which absorbs light of visible wavelengths, UV wavelengths or IR wavelengths.

The term “dye,” as used herein, refers to a soluble, coloring substance which contains a chromophore.

The term “electron dense group,” as used herein, refers to a group which scatters electrons when irradiated with an electron beam. Such groups include, but are not limited to, ammonium molybdate, bismuth subnitrate, cadmium iodide, carbohydrazide, ferric chloride hexahydrate, hexamethylene tetramine, indium trichloride anhydrous, lanthanum nitrate, lead acetate trihydrate, lead citrate trihydrate, lead nitrate, periodic acid, phosphomolybdic acid, phosphotungstic acid, potassium ferricyanide, potassium ferrocyanide, ruthenium red, silver nitrate, silver proteinate (Ag Assay: 8.0-8.5%) “Strong”, silver tetraphenylporphin (S-TPPS), sodium chloroaurate, sodium tungstate, thallium nitrate, thiosemicarbazide (TSC), uranyl acetate, uranyl nitrate, and vanadyl sulfate.

The term “energy transfer agent,” as used herein, refers to a molecule which either donates or accepts energy from another molecule. By way of example only, fluorescence resonance energy transfer (FRET) is a dipole-dipole coupling process by which the excited-state energy of a fluorescence donor molecule is non-radiatively transferred to an unexcited acceptor molecule which then fluorescently emits the donated energy at a longer wavelength.

The term “moiety incorporating a heavy atom,” as used herein, refers to a group which incorporates an ion of atom which is usually heavier than carbon. In some embodiments, such ions or atoms include, but are not limited to, silicon, tungsten, gold, lead, and uranium.

The term “photoaffinity label,” as used herein, refers to a label with a group, which, upon exposure to light, forms a linkage with a molecule for which the label has an affinity.

The term “photocaged moiety,” as used herein, refers to a group which, upon illumination at certain wavelengths, covalently or non-covalently binds other ions or molecules.

The term “photoisomerizable moiety,” as used herein, refers to a group wherein upon illumination with light changes from one isomeric form to another.

The term “radioactive moiety,” as used herein, refers to a group whose nuclei spontaneously give off nuclear radiation, such as alpha, beta, or gamma particles; wherein, alpha particles are helium nuclei, beta particles are electrons, and gamma particles are high energy photons.

The term “spin label,” as used herein, refers to molecules which contain an atom or a group of atoms exhibiting an unpaired electron spin (i.e. a stable paramagnetic group) that in some embodiments are detected by electron spin resonance spectroscopy and in other embodiments are attached to another molecule. Such spin-label molecules include, but are not limited to, nitryl radicals and nitroxides, and in some embodiments are single spin-labels or double spin-labels.

The term “quantum dots,” as used herein, refers to colloidal semiconductor nanocrystals that in some embodiments are detected in the near-infrared and have extremely high quantum yields (i.e., very bright upon modest illumination).

One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering moiety, such as a bivalent saturated or unsaturated hydrocarbon chain.

“Detectably labeled” means a molecule that is associated with a detectable group, e.g., a molecule that is covalently bound to a detectable group.

The term “carbohydrate” refers to a sugar or polymer of sugars. The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

As used herein, excluding uses to denote the point of attachment of a moiety, a “ [symbol] ” bond refers to unspecified stereochemistry (i.e., either of two stereoisomers at that position, or a mixture thereof).

A wide variety of labels may be appropriate to the compositions and methods of the present invention. In certain embodiments the label is not fluorescent and is detectable by other means. In some instances, the substrate is not labeled and is detectable by other means, for example by pulsed amperometric detection of the carbohydrate leaving group, conductivity detection of sulfate leaving group, or by mass spectrometry.

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “acyl,” used alone or a part of a larger moiety, refers to groups formed by removing a hydroxy group from a carboxylic acid.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “halogen” means F, Cl, Br, or I.

The term “aryl” used alone or as part of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “aralkyl” and “arylalkyl” are used interchangeably and refer to alkyl groups in which a hydrogen atom has been replaced with an aryl group. Such groups include, without limitation, benzyl, cinnamyl, and dihyrocinnamyl.

The term “heteroaliphatic,” as used herein, means aliphatic groups wherein one or two carbon atoms are independently replaced by one or more heteroatoms. Heteroaliphatic groups may be substituted or unsubstituted, branched or unbranched, cyclic or acyclic, and include “heterocycle,” “hetercyclyl,” “heterocycloaliphatic,” or “heterocyclic” groups.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may, when specified, contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SRO, —(CH₂)₀₋₄OC(O)NR^{∘}₂; —C(O)N(OR)R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)O₂R^{}, -(haloR^{}), —(CH₂)₀₋₂OH, —(CH₂)O₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}3, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₄ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “suitable” salt refers to any salt that may be formed from a compound described herein. Methods of preparing salts are known in the art, and the skilled artisan is aware of how to select and make salts of compounds described herein. In some embodiments, a suitable salt is formed under appropriate conditions or at physiological pH and may be represented by the removal of one or more hydrogens from acidic groups without showing respective counterions. In some embodiments, a suitable salt is a “pharmaceutically acceptable salt.”

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. In particular embodiments, phosphate and sulfate are not used. It is to be appreciated that certain compositions can inhibit the activity of I2S enzyme. Inhibitory compositions are known in the art.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary, tertiary, or quaternary amine. Salts derived from appropriate bases include alkali metal, alkaline earth metal, ammonium and

N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Representative organic amines useful for the formation of base addition salts include ethylamine, diethylamine, ethylenediamine, ethanolamine, diethanolamine, piperazine and the like. (See, for example, Berge et al., supra).

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or 14C-enriched carbon are within the scope of this invention. Such compounds are useful, for example, as analytical tools, as probes in biological assays, or as therapeutic agents in accordance with the present invention.

The term “oxo,” as used herein, means an oxygen that is double bonded to a carbon atom, thereby forming a carbonyl.

One of ordinary skill in the art will appreciate that the synthetic methods, as described herein, utilize a variety of protecting groups. By the term “protecting group,” as used herein, it is meant that a particular functional moiety, e.g., O, S, or N, is masked or blocked, permitting, if desired, a reaction to be carried out selectively at another reactive site in a multifunctional compound. Suitable protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In certain embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers); and the protecting group will preferably have a minimum of additional functionality to avoid further sites of reaction. As detailed herein, oxygen, sulfur, nitrogen, and carbon protecting groups may be utilized. By way of non-limiting example, hydroxyl protecting groups include methyl, methoxylmethyl (MOM), methylthiomethyl (MTM), benzyloxymethyl (BOM), p-methoxybenzyloxymethyl (PMBM), t-butoxymethyl, siloxymethyl, 2-methoxyethoxymethyl (MEM), 2,2,2-trichloroethoxymethyl, tetrahydropyranyl (THP), 4-methoxytetrahydropyranyl (MTHP), 1-methyl-1-methoxyethyl, 1-methyl-1-benzyloxyethyl, 2-trimethylsilylethyl, allyl, p-chlorophenyl, p-methoxyphenyl, 2,4-dinitrophenyl, benzyl, p-methoxybenzyl, 3,4-dimethoxybenzyl, p-nitrobenzyl, 2,6-dichlorobenzyl, p-phenylbenzyl, 4-picolyl, diphenylmethyl, p,p′-dinitrobenzhydryl, triphenylmethyl, p-methoxyphenyldiphenylmethyl, 1,1-bis(4-methoxyphenyl)-1′-pyrenylmethyl, trimethylsilyl (TMS), triethylsilyl (TES), triisopropylsilyl (TIPS), dimethylisopropylsilyl (IPDMS), dimethylthexylsilyl, t-butyldimethylsilyl (TBDMS), t-butyldiphenylsilyl (TBDPS), triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, pivaloate, adamantoate, crotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, o-(dibromomethyl)benzoate, 2-(methylthiomethoxy)ethyl, 2-(methylthiomethoxymethyl)benzoate, 2,6-dichloro-4-methylphenoxyacetate, 2,6-dichloro-4-(1,1,3,3-tetramethylbutyl)phenoxyacetate, chlorodiphenylacetate, isobutyrate, monosuccinoate, o-(methoxycarbonyl)benzoate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described by Greene and Wuts (supra).

Glycosaminoglycans (GAGs) are a class of polysaccharides that bind to a wide variety of proteins and signaling molecules in the cellular environment and, in some instances, modulate their activity, sometimes impinging on normative biological processes. GAGs can be linear acidic polysaccharides containing disaccharide repeat units of an uronic acid linked to a hexosamine, and there are at least four classes of GAGs based on the different chemical structures. In some instances, GAG backbones can be modified by sulfation at the uronic acid and hexosamine. For example, heparan sulfate glycosaminoglycans (HSGAGs) can potentially contain up to 48 disaccharide building blocks based on the sulfation pattern.

Heparin-like glycosaminoglycans (HLGAGs) are complex polysaccharides that may, in some instances, be characterized by a disaccharide repeat unit of a uronic acid (either L-iduronic acid or D-glucuronic acid) which is linked 1-4 to a glucosamine. The modification of the functional groups of the sugar units (i.e., 2-0 sulfate on the uronic acid and 3-0, 6-0, and N-sulfation of the hexosamine), taken together with the variation in the chain length, contribute to the heterogeneity of HLGAGs.

In various embodiments, a physiologically relevant I2S substrate, e.g., an iduronate-2-sulfate, includes a sulfate and one or more free or unprotected hydroxyl groups.

### Methods for the Determination of Kinetic Parameters or Specific Activity

Various methods may be used to determine kinetic parameters or specific activity of I2S enzymes of the present invention. For example, various kinetic models are known in the art and can be used to determine kinetic parameters or specific activity. As used herein, the term “kinetic model” refers to any quantitative description of enzyme reaction rate. Typically, a kinetic model provides a rate equation and/or time course of the reaction. For example, a Michaelis-Menten kinetic model is a common model of a single-substrate reaction. As used herein, kinetic parameters include any parameters indicative of reaction rate and specific activity. Exemplary kinetic parameters with exemplary units for each kinetic parameters include, but are not limited to, Vₘₐₓ (μM/min), Kₘ (μM), k_{cat} (s⁻¹). For example, Vₘₐₓ represents the maximum rate achieved by the system, at maximum (saturating) substrate concentrations. Typically, enzyme-catalyzed reactions are saturable. Their rate of catalysis does not always show a linear response to increasing substrate. If the initial rate of the reaction is measured over a range of substrate concentrations (denoted as [S]), the reaction rate (v) generally increases as [S] increases. However, as [S] gets higher, the enzyme becomes saturated with substrate and the rate reaches Vₘₐₓ, the enzyme's maximum rate. Kₘ, also known as the Michaelis constant, is the substrate concentration at which the reaction rate is half of Vₘₐₓ. Specific activity is typically defined as the amount of substrate the enzyme converts (reactions catalyzed), per mg protein in the enzyme preparation, per unit of time. The range of any particular parameter as determined, e.g., by a method of the present invention or by a method including a composition of the present invention, will vary depending upon numerous factors and conditions.

In certain embodiments of the present invention, kinetic parameters or specific activity of I2S enzyme are determined by incubating an I2S enzyme sample with a desired amount of substrate under conditions that permit I2S to catalyze desulfation of the substrate and analyzing the formation of one or more products. Thus, various assay reactions can occur under conditions that permit of I2S-catalyzed desulfation of a physiologically relevant substrate. In some instances, the reaction mixture can be separated by chromatography. After chromatography, a detection unit can be used to measure the product signal. In some instances, a product will include a detectable label. In various embodiments, it is critical that the catalytic reaction is in the initial rate region (where the product formation or substrate depletion is linear with respect to time); in such embodiments, only under initial rate conditions is the Michaelis-Menten model valid for determining kinetic parameters. Therefore, in such embodiments, experimental conditions must be selected to ensure that initial rates are measured, and to ensure that all other Michaelis-Menten model assumptions are met.

An I2S enzyme of the present invention may be any I2S enzyme as described herein. The I2S enzyme may be provided at, or diluted to, a concentration of 0.001 mg/mL or more, e.g., 0.001 mg/mL, 0.005 mg/mL, 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, or more, or any range therebetween. The I2S enzyme may alternatively be provided at, or diluted to, a concentration of 0.001 μg/mL or more, e.g., 0.001 μg/mL, 0.005 μg/mL, 0.01 μg/mL, 0.02 μg/mL, 0.03 μg/mL, 0.04 μg/mL, 0.05 μg/mL, 0.1 μg/mL, 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, or more, or any range therebetween. The enzyme concentration may also be less than 0.001 μg/mL. In some instances, the amount of enzyme in a sample is unknown. For instance, in some instances a sample may be tested based on the mass or volume of starting sample rather than any determination of the composition of the sample with respect to enzyme. Those of skill in the art will appreciate that the concentration of a molecule, when provided as a mass per unit volume, is equivalent to providing that molecules molarity when the mass of the molecule is known. In particular instances, enzyme concentration may be based on a molecular mass of about 70 to about 80 kDa, e.g., about 70, 71, 72, 73, 74, 75, 76, 77, 78, 79, or 80 grams, e.g., 76 kDa or 78.8 kDa (˜g/mol) as determined by a number of approaches, e.g., mass spectrometry.

An I2S substrate of the present invention may be any I2S substrate as described herein. The I2S substrate may be provided at, or diluted to, a concentration of 0.1 μM or more, such as 0.1 μM, 0.2 μM, 0.3 μM, 0.4 μM, 0.5 μM, 1 μM, 2 μM, 3 μM, 4 μM, 5 μM, 10 μM, 15 μM, 20 μM, 25 μM, 30 μM, 35 μM, 40 μM, 45 μM, 50 μM, 100 μM, 200 μM, 300 μM, 400 μM, 500 μM, 1 mM, 2 mM, 3 mM, 4 mM, 5 mM, 10 mM, or higher, or any range therebetween. The substrate concentration may also be less than 0.1 μM. The assay of the present invention may occur in wells appropriate to the volume of the assay reaction. The reaction volume may be, for example, 1 μL, 2 μL, 3 μL, 4 μL, 5 μL, 10 μL, 20 μL, 30 μL, 40 μL, 50 μL, 100 μL, 200 μL, 300 μL, 400 μL, 500 μL, 1 mL, 2 mL, 3 mL, 4 mL, 5 mL, 10 mL, or more, or any range therebetween.

A single assay reaction may include, for example, 0.01 ng or more of I2S enzyme, such 0.01 ng, 0.02 ng, 0.03 ng, 0.04 ng, 0.05 ng, 0.1 ng, 0.2 ng, 0.3 ng, 0.4 ng, 0.5 ng, 1 ng, 2 ng, 3 ng, 4 ng, 5 ng, 10 ng, or more I2S enzyme. A single assay reaction may further include, 0.1 or more nanomoles of substrate. For example, a single assay reaction may include 0.1 nanomoles, 0.2 nanomoles, 0.3 nanomoles, 0.4 nanomoles, 0.5 nanomoles, 1 nanomole, 2 nanomoles, 3 nanomoles, 4 nanomoles, 5 nanomoles, 6 nanomoles, 7 nanomoles, 8 nanomoles, 9 nanomoles, 10 nanomoles, 50 nanomoles, 100 nanomoles, 200 nanomoles, or more substrate, or any range therebetween.

The ratio of enzyme to substrate in an assay reaction may range from, e.g., 100 ng enzyme per 0.01 nanomoles of substrate to 0.01 ng enzyme per 100 nanomoles of substrate. For instance, the ratio of enzyme to substrate may be 100 ng enzyme per 0.01 nanomoles of substrate, 75 ng enzyme per 0.01 nanomoles of substrate, 50 ng enzyme per 0.01 nanomoles of substrate, 25 ng enzyme per 0.01 nanomoles of substrate, 1 ng enzyme per 0.01 nanomoles of substrate, 0.1 ng enzyme per 0.01 nanomoles of substrate, 0.05 ng enzyme per 0.01 nanomoles of substrate, 0.01 ng enzyme per 0.01 nanomoles of substrate, 0.01 ng enzyme per 0.01 nanomoles of substrate, 0.01 ng enzyme per 0.05 nanomoles of substrate, 0.01 ng enzyme per 0.1 nanomoles of substrate, 0.01 ng enzyme per 1 nanomole of substrate, 0.01 ng enzyme per 25 nanomoles of substrate, 0.01 ng enzyme per 50 nanomoles of substrate, 0.01 ng enzyme per 75 nanomoles of substrate, 0.01 ng enzyme per 100 nanomoles of substrate, 100 ng enzyme per 0.01 nanomoles of substrate, 75 ng enzyme per 0.05 nanomoles of substrate, 50 ng enzyme per 0.1 nanomoles of substrate, 25 ng enzyme per 1 nanomoles of substrate, 1 ng enzyme per 25 nanomoles of substrate, 0.1 ng enzyme per 50 nanomoles of substrate, 0.05 ng enzyme per 75 nanomoles of substrate, 0.01 ng enzyme per 100 nanomoles of substrate. In certain assay reactions, the ratio of enzyme to substrate may be, e.g., 1 ng enzyme per 0.1 nanomoles substrate or 0.1 ng enzyme per 1 nanomole substrate, or any range therebetween. An assay reaction may further include any ratio of enzyme to substrate not otherwise described herein. In certain embodiments, the concentration of substrate is significantly greater than the concentration of enzyme. In such embodiments, a concentration of substrate greater than the concentration of enzyme can facilitate application of the Micahelis-Menten model.

Upon mixing of substrate and enzyme, the assay reaction may be incubated for a period sufficient to allow the enzyme to act upon the substrate in a detectable manner, preferably, in some instances, while maintaining the product formation in the initial rate region. Controls and standards, when present, should be incubated in kind. In certain instances, the reaction is incubated at a temperature between 1° C. and 99° C., such as 10° C., 15° C., 20° C., 25° C., 30° C., 35° C., 40° C., 45° C., or 50° C., or any range therebetween. In particular instances, the reaction may be incubated at a temperature between 15° C. and 45° C., e.g., a temperature between 20° C. and 37° C., a temperature between 25° C. and 30° C., a temperature between 35° C. and 40° C., at room temperature, at 37° C., a temperature between 32° C. and 42° C., e.g., 32° C., 33° C., 34° C., 35° C., 36° C., 37° C., 38° C., 39° C., 40° C., 41° C., or 42° C.

In some instances the pH used in the present assay may be acidic. The pH may be acidic for one or more or all of control assay reactions, experimental assay reactions, or standard curve assay reactions. For instance, assay reactions may be at a pH of 1 to 6.5, such as 1, 1.5, 2, 2.5, 3, 3.5, 4, 4.5, 5, 5.5, 6, or 6.5, or any range therebetween. In other instances one or more assay reactions may have a neutral or basic pH, such as a pH of 6.5 to 7.5 or a pH of 7.5 to 14.

Assay reactions may further include BSA. For instance, assay reactions may include BSA at a concentration of 0.001 to 1 mg/mL, e.g., 0.001, 0.005, 0.01, 0.05, 0.1, 0.2, 0.3, 0.4, 0.5, 0.6, 0.7, 0.8, 0.9, or 1 mg/mL, or any range therebetween. In particular instances, assay reactions may include between 0 and 0.4 mg/mL BSA. Various kinetic parameters or specific activity as determined by such assays may depend in part upon the concentration of BSA.

The length of the incubation period may be from 10 seconds to 2 weeks or longer, such as 10 second, 30 seconds, 1 minute, 5 minutes, 10 minutes, 20 minutes, 30 minutes, 40 minutes, 50 minutes, 1 hour, 2 hours, 3 hours, 4 hours, 5 hours, 6 hours, 12 hours, 1 day, 2, days, 3 days, 4 days, 5 days, 6 days, 1 week, 2 weeks, or longer. The incubation temperature may modulate the appropriate period of time for the incubation of the reaction. In various embodiments, the length of the incubation period is within the initial rate region.

Following the incubation period, the enzymatic reaction may be quenched through the addition of a quenching agent. An exemplary quenching agent is acetonitrile. Acetonitrile may be provided in a pure form or in a diluted form, e.g., a dilution that is 95%, 90%, 85%, 80%, 75%, 70%, 65%, 60%, 55%, 50%, 45%, 40%, 35% or 30% or less acetonitrile by volume. In particular instances, the acetonitrile may be diluted in water. Other quenching agents that may be used alternatively or in combination with acetonitrile are known in the art. For instance, heat inactivation or quenching by one or more of methanol, ethanol, isopropyl alcohol, acetone, or the like, or organic solvents in general, are also contemplated herein.

Quenched samples may be optionally filtered using a protein precipitation apparatus such as a 96-well protein precipitation plate. Protein precipitation apparatuses are known in the art as are the method appropriate to their use. Other methods of filtering may be applied as known in the art. For instance, quenched samples may be filtered through a 0.2 m filter and/or centrifuged to remove precipitated protein.

Kinetic parameters or specific activity of an I2S enzyme can be determined using any of a variety of apparatuses in accordance with the detectable group of the utilized substrate. In particular instances, the qualification or quantification of kinetic parameters or specific activity relating to an I2S enzyme may be determined by a method including a step in which substrate that has been acted upon by I2S enzyme (i.e., product) and substrate that has not been acted upon by I2S enzyme (i.e., substrate). Samples may be separated, e.g., by any of the chromatographic methods provided herein.

In particular instances, the method of separation may include liquid chromatography, thin-layer chromatography, capillary electrophoresis, gas chromatography, or solvent extraction. In some instances, the method of separation may include adsorption chromatography, partition chromatography, normal-phase chromatography, aqueous normal phase chromatography, reverse-phase chromatography, ion exchange chromatography, molecular or size exclusion chromatography, or affinity chromatography. The method of detection may include ultra-performance liquid chromatography (UPLC) or high-performance liquid chromatography (HPLC). HPLC is a method in which a pressurized liquid solvent is contacted with a support, such as a column, the characteristics of which can mediate the separation of molecules present in a mixture. UPLC is a variant of HPLC that may include particle sizes smaller than those used in traditional HPLC methods (e.g., less than 2 um) and may utilize higher pressures than traditional HPLC methods. Methods of HPLC and UPLC are known in the art. A method of detection including chromatography may include a hydrophilic interaction liquid chromatography (HILIC), reversed phase (RP), or charged surface hybrid (CSH) column. In some instances, separation will include one or more steps in which molecules are distinguished based on, e.g., size, polarity, hydrophobicity, charge, fluorescence, radioactivity, spectrophotometric characteristics, spectra, mass, or other characteristics known in the art, or any combination thereof.

Chromatography may include a first eluent A (10% acetonitrile, 20 mM ammonium formate, pH 3.5; prepared from 800 mL Milli-Q water, 100 mL of 200 mM Ammonium formate, pH 3.5, and 100 mL acetonitrile), and a second eluent B (90% acetonitrile, 20 mM ammonium formate, pH 3.5; prepared from 100 mL of 200 mM ammonium formate, pH 3.5 and 900 mL acetonitrile) with a flow rate of 0.3 mL/min and an isocratic gradient of 93% B. Sampling can be configured for 3 μL injections of sample and a 10 minute run time. The column temperature can be 40° C.+/−5° C., while the autosampler temperature can be 4° C.+/−2.5° C. Fluorescence detection, e.g., for 4-MU labeled substrate, can occur with an excitation wavelength of 308 nm and an emission wavelength of 370 nm.

Separated or unseparated samples may be subjected to a detection step. For instance, fluorescence detection can be useful for sensitive, precise, and/or accurate quantitation at low levels of analyte. In instances in which the substrate includes a detectable group capable of producing a fluorescent signal, corresponding methods of detection may include the use of a fluorometer or spectrofluorometer, fluorescence plate reader, fluorescence microscopes, fluorescence scanners, or flow cytometers. The fluorescence of the detectable group is determined for one or more samples or a portion of one or more samples, such as a portion including product that has been separated from substrate.

In certain embodiments in which the substrate does not contain a detectable group capable of producing a fluorescent signal, or in embodiments in which the substrate does not contain a detectable group, applicable methods of detection are known in the art. Methods of detection suitable to substrates without a fluorescent detectable group or without any detectable group include, without limitation, conductivity detection to detect sulfate release, pulsed amperometric detection to detect the substrate and product carbohydrates, as well as other methods known in the art and/or described herein.

In certain embodiments, the method includes a separation step and a detection step. In particular instances, chromatographic separation is performed in conjunction with downstream fluorescence or conductivity detection. Without limiting the scope of the present invention, specific examples include the use of ultra-performance liquid chromatography coupled to fluorescence detection (UPLC-FLD). Apparatuses and techniques for UPLC, FLD, and UPLC-FLD are known in the art. For instance, a Waters Acquity I-Class UPLC with fluorescence detection can be used in conjunction with a BEH amide column, 1.7 μm, 2.1×100 mm. Other methods of detection may be applied in conjunction or in place of fluorescence detection, including methods of distinguishing molecules based, e.g., mass, size, charge, ionic character, or conductivity. Conductivity can be detected with or without the use of suppression. When used in combination with a suppressor system, a conductivity detector can be a solute-specific detector. Conductivity detectors can be used in combination with ion chromatography. Conductivity detectors include, e.g., the ICS-5000+ CD Conductivity Detector, the D50A Electrochemical Detectors with conductivity cell and DS3 Detectors Stabilizer, the Waters 432 Conductivity Detector for HPLC Systems, the Shimadzu CDD-10AVP conductivity detector, and other detectors known in the art. In particular instances, the invention includes, e.g., ion chromatography with conductivity detection (sulfate release).

Assays to determine kinetic parameters or specific activity of I2S enzyme can include control reactions. A control reaction may include substrate from a stock or formulation of substrate previously shown to be acted upon by I2S, I2S enzyme from a stock or formulation of enzyme previously shown to be capable of acting upon an I2S substrate, or both. An assay to determine kinetic parameters or specific activity of I2S enzyme can further include wells that include enzyme without substrate, substrate without enzyme, or product without substrate or enzyme. In particular, a standard curve may be generated using wells having a range of concentrations of product. In certain instances, neither enzyme nor substrate is added to wells used to produce a standard product curve. The standard substrate curve facilitates the correlation of assay readouts with concentrations of product.

Various embodiments of the present invention utilize concentration ranges of substrate or product, as appropriate, in control, experimental, and standard curve wells. For instance, a substrate control may be tested across a range of substrate concentrations; a substrate control may be tested across a range of enzyme concentrations; an enzyme control may be tested across a range of enzyme concentrations; an enzyme control may be tested across a range of substrate concentrations; and/or a standard curve may be constructed across a range of product concentrations. Applicable controls may vary depending upon whether the experimental assay reactions include a known substrate and an unknown enzyme, an unknown substrate and a known enzyme, or an unknown substrate and an unknown enzyme. In certain instances, a single assay includes two, three, four, or more replicates of each control, experimental, and standard curve condition.

In certain methods of determining kinetic parameters or specific activity of an I2S enzyme, a product standard curve provides the basis for determining the concentration of IdoA-4MU in each assay reaction or aliquots thereof, allowing the rate of product formation to be plotted against substrate concentration. For instance, an IdoA-4MU product standard curve can be generated by first calculating the average peak area for IdoA-4MU product standard concentration. Subsequently, a linear regression curve of the average peak area vs. the IdoA-4MU product standard concentration (μM) can be generated using, e.g., an Empower processing method or Excel. Characteristics of the linear regression curve can be determined, such as R² values and % CVs. Velocities can be calculated from the product peak areas and the incubation time.

For each injection of assay control and sample at each substrate concentration, the peak area can be converted to a concentration using the standard curve parameters and divided by 20 minutes to obtain the velocity (μM/min). The average velocity and % CV of the triplicate determination for each substrate concentration were determined and recorded.

Methods of deriving particular kinetic parameters or specific activity from such data are known in the art. For instance, the data can fitted to the Michaelis-Menten model to obtain Vₘₐₓ (μM/min) and Kₘ (μM). Moreover, k_{cat} (s⁻¹) can be calculated by dividing Vₘₐₓ with the enzyme concentration [E] (μM). Values of k_{cat} and Kₘ can be determined by performing non-linear regression utilizing each replicate velocity and the corresponding substrate concentration. Non-linear regression fit can be performed using the Michaelis-Menten equation: v₀=Vₘₐₓ[S]/(Kₘ+[S]). Vₘₐₓ can be divided by the total enzyme concentration in the reaction and divided by 60 seconds/minute to obtain k_{cat}(s⁻¹). In certain instances, the enzyme concentration used to calculate k_{cat} assumes 100% formylglycine and 100% correctly folded active sites that are catalytically competent. In certain instances, enzyme concentration in a reaction, as used for calculating k_{cat} from Vₘₐₓ, may be based on a molecular mass of about 70 to about 80 kDa, e.g., about 70, 71, 72, 73, 74, 75, 76, 77, 78, 79, or 80 grams, e.g., 76 kDa or 78.8 kDa (˜g/mol) as determined by a number of approaches, e.g., mass spectrometry. The determination of enzyme kinetic parameters or specific activity may utilize software capable of performing non-linear regression. The k_{cat} (s⁻¹) and Kₘ (μM) of the non-linear regression fit are thereby determined. These and other kinetic parameters known in the art, or specific activity, may be determined by methods of calculation known in the art. For instance, the Hill equation including the Hill coefficient (n; Vₘₐₓ*Sⁿ/(Kₘⁿ+Sⁿ)) may be used to calculate kinetic parameters such as Vₘₐₓ, Kₘ, n, and k_{cat} (from Vₘₐₓ). In particular instances the formula Vₘₐₓ/[1+Kₘ/S+S/Ki] can be used to calculate kinetic parameters such as Vₘₐₓ, Kₘ, Kᵢ, and k_{cat} (from Vₘₐₓ).

The present compositions and methods for determining I2S enzyme kinetic parameters or specific activity, in some instances, have sensitivity in the very low numbers of attomoles. In some instances, the present invention enables the detection of I2S enzyme activity from biological samples with I2S concentrations of 0.001 mg/mL or more, such as 0.001 mg/mL, 0.005 mg/mL, 0.01 mg/mL, 0.05 mg/mL, 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 35 mg/mL, 40 mg/mL, 45 mg/mL, 50 mg/mL, or more, or any range therebetween. In some instances, the amount of enzyme detected can be measured in U/mg, where U is the amount of enzyme required to catalyze the desulfation of 1 μmol of substrate per minute. For instance, present compositions and methods for determining kinetic parameters or specific activity of I2S enzyme can, in some instances, detect 0.1 U/mg I2S enzyme or more, such as 0.1 U/mg, 0.2 U/mg, 0.3 U/mg, 0.4 U/mg, 0.5 U/mg, 1 U/mg, 2 U/mg, 3 U/mg, 4 U/mg, 5 U/mg, 6 U/mg, 8 U/mg, 10 U/mg, 12 U/mg, 14 U/mg, 16 U/mg, 18 U/mg, 20 U/mg, 22 U/mg, 24 U/mg, 26 U/mg, 28 U/mg, 30 U/mg, 40 U/mg, 50 U/mg, 60 U/mg, 70 U/mg, 80 U/mg, 90 U/mg, 100 U/mg, or more U/mg I2S enzyme, or any range therebetween.

The present compositions and methods for determining kinetic parameters of I2S enzyme can, in some instances, detect a Kₘ value of 0.1 μM or more, such as 0.1 μM, 0.5 μM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, 100 μM, 200 μM, 300 μM, 400 μM, 500 μM, 1 mM, or more, or any range therebetween. In particular instances, 95% (2 standard deviations) of a set of Kₘ values fall between the range of 0.1 μM and 500 μM, 1 μM and 200 μM, 5 μM and 100 μM, 10 μM and 100 μM, or 11 μM and 73 μM.

The present compositions and methods for determining kinetic parameters of I2S enzyme can, in some instances, detect a k_{cat} value of 0.1 s⁻¹ or more, such as 0.1 s⁻¹, 0.5 s⁻¹, 1 s⁻¹ 2 s⁻¹, 3 s⁻¹, 4 s⁻¹, 5 s⁻¹, 6 s⁻¹, 7 s⁻¹, 8 s⁻¹, 9 s⁻¹, 10 s⁻¹, 15 s⁻¹, 20 s⁻¹, 25 s⁻¹, 30 s⁻¹, 35 s⁻¹, 40 s⁻¹, 45 s⁻¹, 50 s⁻¹, 60 s⁻¹, 70 s⁻¹, 80 s⁻¹, 90 s⁻¹, 100 s⁻¹, 200 s⁻¹, 300 s⁻¹, 400 s⁻¹, 500 s⁻¹, 1000 s⁻¹, or more, or any range therebetween. In particular instances, 95% (2 standard deviations) of a set of k_{cat} values fall between the range of 0.1 s⁻¹ and 500 s⁻¹, 1 s⁻¹ and 200 s⁻¹, 5 s⁻¹ and 100 s⁻¹, 10 s⁻¹ and 50 s⁻¹, or 14 s⁻¹ and 34 s⁻¹.

### Applications

The methods and compositions of the present invention may be employed toward a variety of applications. For instance, methods and compositions of the present invention can be used to monitor manufacturing and purification processes.

In particular, the present invention includes a method for assessing clinically relevant properties of I2S enzyme for use in enzyme replacement therapy. For example, kinetic parameters or specific activity determined according to the present invention may be indicative of enzyme potency; thus, can be used as a surrogate of efficacy of I2S for therapeutic use.

The present invention may also be used to as quality control during manufacturing process. For instance, commercial production of I2S enzyme therapeutics may include the production of independent, semi-independent, differently or separately treated, or differently or separately handled batches, samples, or aliquots of I2S enzyme or I2S enzyme therapeutic. In such instances, samples of I2S enzyme from diverse sources may be tested to ensure that the I2S enzyme from the various sources possesses consistent or substantially consistent kinetic parameters or specific activity, or kinetic parameters or specific activity sufficiently consistent for therapeutic purposes. In some instances, kinetic parameters or specific activity may differ and the production of therapeutic using I2S enzyme from one or more particular sources may be adjusted accordingly with reference to an established standard or therapeutic target.

Further applications relating to kinetic parameters or specific activity of I2S enzyme can include the determination of kinetic parameters or specific activity of stored I2S enzyme. I2S enzyme can be stored at a variety of temperatures, such as 50° C. or less, e.g., 50° C., 40° C., 30° C., 20° C., 10° C., 0° C., −10° C., −20° C., −30° C., −40° C., −50° C., −100° C. or less. Particular storage temperatures may include, e.g., 2° C., 8° C., −65° C., −80° C., or −85° C. Storage times at any temperature may be, e.g., 1 minute to 6 months, e.g., 1 minute, 30 minutes, 1 hour, 2 hours, 4 hours, 6 hours, 8 hours, 10 hours, 12 hours, 1 day, 2 days, 3 days, 4 days, 5 days, 6 days, 1 week, 2 weeks, 3 weeks, 1 month, 2 months, 3 months, 6 months, or longer. Storage times may be longer for stabilized compositions such as stabilized therapeutic compositions. The length of storage may be determined in accordance with the storage temperature or other storage conditions. For instance, in some embodiments, I2S enzyme may be stored at 25° C. for 8 hours or at 2° C. or less for more than 24 hours. Kinetic parameters or specific activity of I2S enzymes may be determined over the course of storage to ensure sufficient maintenance of enzyme function. For instance, kinetic parameters or specific activity of stored I2S enzyme may be sampled at a single interval or at multiple intervals at or at the frequency of 1 minute to 6 months, e.g., 1 minute, 30 minutes, 1 hour, 2 hours, 4 hours, 6 hours, 8 hours, 10 hours, 12 hours, 1 day, 2 days, 3 days, 4 days, 5 days, 6 days, 1 week, 2 weeks, 3 weeks, 1 month, 2 months, 3 months, 6 months, or longer as measured from the beginning of the storage period. Similarly, methods of the present invention may be used to evaluate kinetic parameters or specific activity of stored I2S substrate.

Additional applications of the present invention may include use in methods of diagnostics or personalized medicine. For instance, a sample of I2S taken from a subject may be used to determine the activity of I2S enzyme from that subject. In such instances, the sample may be used in its initial form or may be further processed, e.g., to purify I2S or separate distinct forms of I2S that may be present in the sample. A specific activity or level of one or more I2S kinetic parameters below a predetermined standard or disease threshold may indicate that treatment with I2S enzyme should be recommended or undertaken. Moreover, determined kinetic parameters or specific activity of subject I2S enzyme may be used to determine the dosage or form of I2S enzyme to be recommended, prescribed, or administered as a therapeutic. For instance, where the activity of I2S enzyme taken from a subject having, at risk of having, or having been diagnosed with an I2S enzyme deficiency condition, such as Hunter Syndromes, is known, a therapeutic formulation or dosage may be prescribed in a manner corresponding to or compensatory for the degree of deficiency. As such, a therapeutic dosage of I2S enzyme may be provided in a manner that compensates for an I2S enzyme or enzyme activity deficiency in a degree commensurate with deficiency, e.g., as the deficiency relates to a predetermined standard or disease threshold.

In addition, certain studies have encountered difficulty in determining genotype-phenotype correlations between the gene encoding the I2S enzyme, or the protein sequence of the I2S enzyme produced by the gene, and phenotypes associated with I2S enzyme deficiency. For instance, challenges may be encountered in distinguish severe and attenuated forms of Hunter Syndrome based on previously available measures of I2S enzyme kinetic parameters or specific activity. Moreover, in subjects having been therapeutically treated with I2S enzyme, samples of I2S may be taken from the subject and I2S enzyme kinetic parameters or specific activity of the I2S enzyme in the sample may be determined according to the present invention in order to determine the efficacy or stability of treatment and related treatment parameters. The presently claimed compositions and methods may provide greater diagnostic capacity for these and related applications.

### Kits

The present invention includes kits for the determination of I2S specific activity or one or more I2S enzyme kinetic parameters. In particular, certain kits of the present invention may include one or more of eluent A (10% acetonitrile, 20 mM ammonium formate, pH 3.5), eluent B (90% acetonitrile, 20 mM ammonium formate, pH 3.5), and a UPLC-FLD apparatus. Kits of the present invention may further include instructions for the use of the kit in determining kinetic parameters or specific activity of I2S enzyme. Components of the present invention or components required for the operation of the present invention may also be provided in a compact unit or portable device such as a table top, miniaturized, or hand-held device.

The invention will be more fully understood by reference to the following examples. They should not, however, be construed as limiting the scope of the invention. All literature citations are incorporated by reference.

### EXAMPLES

The examples described herein demonstrate the use of a physiologically relevant substrate as a reagent in the determination of kinetic parameters of enzymatically catalyzed desulfation and demonstrate determination of enzyme specific activity. In particular, certain of the below examples demonstrate the use of a physiologically relevant substrate containing a terminal iduronate-2-sulfate, in particular 4-methylumbelliferyl-α-L-idopyranosiduronic acid 2-sulfate (IdoA2S-4MU), to determine kinetic parameters or specific activity of idursulfase enzyme (iduronate-2-sulfatase, I2S). I2S is capable of catalyzing the desulfation of the substrate IdoA2S to generate iduronate (IdoA) and sulfate (FIG. 1). In particular, desulfation of the substrate IdoA2S-4MU generates 4-methylumbelliferyl-α-L-idopyranosiduronic acid sodium salt and sulfate. Certain of the below examples include a step in which 4-methylumbelliferyl ca-L-idopyranosiduronic acid (IdoA-MU) is hydrolyzed to idopyranosiduronic acid (IdoA) and 4-methylumbelliferone (4-MU). The second step is catalyzed by iduronidase (IDUA), and/or a step in which a high pH quench generates an anionic version of 4-MU that is measurable by fluorescence.

While the below examples are discussed in the context of testing experimental samples of enzyme, the below methods may be readily applied to the testing of various drug substances (DSs), drug products (DPs), or DS and DP stability samples.

Although exemplary embodiments have been described in detail, it should be understood that various changes, substitutions and alterations can be made herein without departing from the spirit and scope of the invention as defined in the appended claims.

### Example 1

### Preparation of Standard Curve Solutions

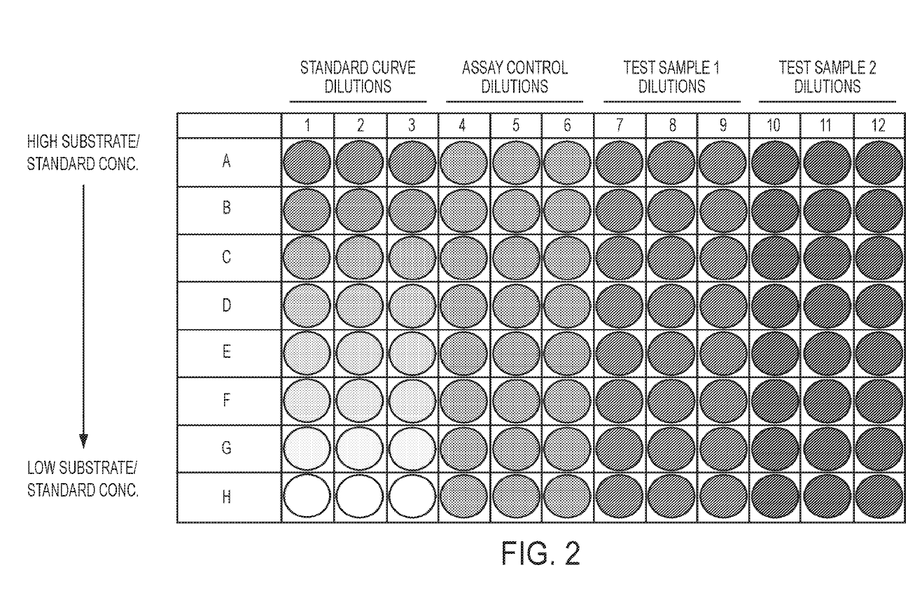

A standard curve was produced using a dilution series of the IdoA-4MU reaction product, which may be produced as a product of the desulfation of IdoA2S-4MU as catalyzed by an idursulfase enzyme. The dilution series was prepared by dilution of 1 mM IdoA-4MU in assay buffer (10 mM sodium acetate, pH 4.5, 0.030 mg/mL BSA). Assay buffer was prepared by gently mixing by inversion 89.5 mL Milli-Q water, 0.6 mL of 5 mg/mL BSA, 10 mL 0.1 M Sodium Acetate Buffer, pH 4.5. A 100 μM solution of IdoA-4MU was prepared by diluting 10 μL of 1 mM IdoA-4MU into 90 μl assay buffer. The solution was vortexed 1 second to mix. Three independent 100 μM dilutions of the IdoA-4MU were produced. Serial dilutions of each of these three replicates were independently produced in a 96-well dilution plate having 1 mL well volumes by mixing IdoA-4MU solution with assay buffer as shown in Table 2, each well of the dilution series containing a total volume of 100 or 200 μl. As shown in FIG. 2, each replicate of the standard curve dilution series occupies one of the first three columns (columns 1-3) of the 96-well dilution plate and is oriented with the highest concentration in row A, with concentration decreasing sequentially toward row H.

TABLE 2

| | Final | | |
|---|---|---|---|
| Position in | Concentration | | Assay |
| Series (row) | (μM) | IdoA-4MU Solution | Buffer |
| A | 10.0 | 20 μL of 100 μM | 180 μL |
| B | 5.0 | 100 μL of 10.0 μM | 100 μL |
| C | 2.50 | 100 μL of 5.00 μM | 100 μL |
| D | 1.25 | 100 μL of 2.50 μM | 100 μL |
| E | 0.625 | 100 μL of 1.25 μM | 100 μL |
| F | 0.313 | 100 μL of 0.625 μM | 100 μL |
| G | 0.156 | 100 μL of 0.313 μM | 100 μL |
| H | 0.0781 | 100 μL of 0.156 μM | 100 μL |

### Example 2

### Preparation of Substrate

Samples were assayed using a dilution series of the substrate IdoA2S-4MU. To produce this serial dilution series, an aliquot of 5 mM IdoA2S-4MU was diluted in assay buffer as shown in Table 3, each well of the dilution series containing a total of 500 μL.

TABLE 3

| Position | Concentration | | Assay |
|---|---|---|---|
| in Series | (μM) | IdoA2S-4MU | Buffer |
| A | 600 | 60 μL of 5.00 mM | 440 μL |
| B | 300 | 250 μL of 600 μM | 250 μL |
| C | 150 | 250 μL of 300 μM | 250 μL |
| D | 75.0 | 250 μL of 150 μM | 250 μL |
| E | 37.5 | 250 μL of 75.0 μM | 250 μL |
| F | 18.8 | 250 μL of 37.5 μM | 250 μL |
| G | 9.38 | 250 μL of 18.8 μM | 250 μL |
| H | 4.69 | 250 μL of 9.38 μM | 250 μL |

### Example 3

### Preparation of Samples

Frozen vials of two experimental samples and one assay control sample (a sample of enzyme previously shown to catalyze the desulfation the substrate IdoA2S by methods similar to those of the present examples), were brought to room temperature. Vials were vortexed for one second and centrifuged using several pulses to collect liquid. Experimental and control samples were diluted to obtain a concentration of 0.20 mg/mL of enzyme. Three independent dilutions of each sample were produced. Each 0.20 mg/mL sample was further diluted to a concentration of 0.020 ng/μL.

From the three independent 0.020 ng/μL dilutions of the assay control sample, 80 L aliquots were transferred to individual wells of columns 4-6 of a 96 well dilution plate as shown in FIG. 2.

Also as shown in FIG. 2, aliquots of each of the three independent 0.020 ng/μL dilutions of a first experimental sample were respectively transferred to columns 7-9 of the dilution plate, while aliquots of each of the three independent 0.020 ng/μL dilutions of a second experimental sample were respectively transferred to columns 7-9 of the dilution plate.

### Example 4

### Enzyme Reaction and Data Collection

Briefly, 8 concentrations of the substrate IdoA2S-4MU (2.3-300 μM final concentrations) were incubated with I2S (0.4 ng) at 37° C. for 20 minutes. Following incubation, the reaction was quenched by the addition of acetonitrile, and the samples were filtered with a 96-well protein precipitation plate. A Sirocco protein precipitation plate or similarly capable apparatus may be used. Filtered reaction mixture was analyzed by ultra-performance liquid chromatography coupled to fluorescence detection (UPLC-FLD).

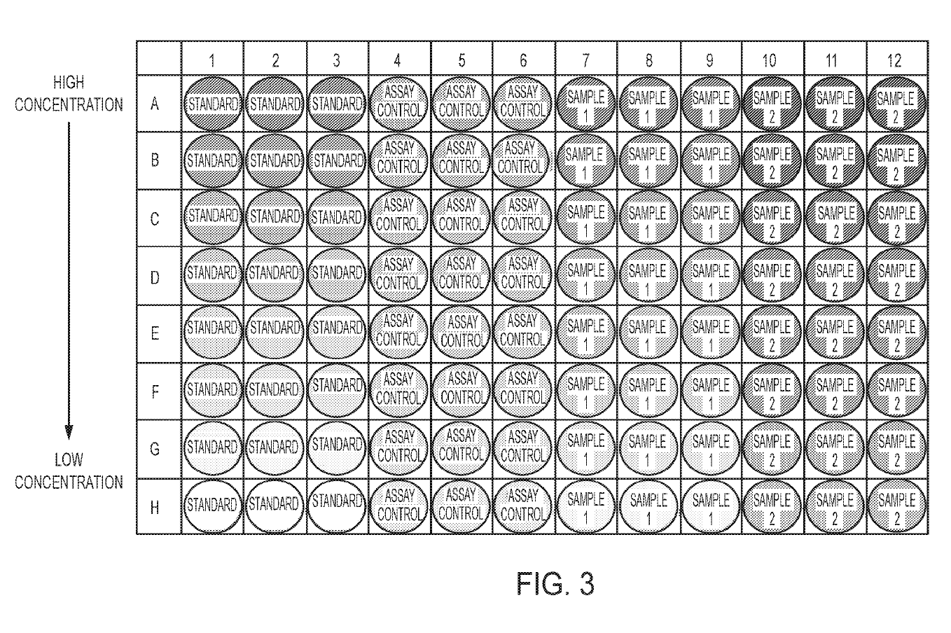

In particular, a 96-well assay plate having columns 1-12 and rows A-H was prepared for the enzyme reaction (FIG. 3). Each of the wells received 40 μL of a standard curve solution (Example 1; columns 1-3) or 20 μL of a substrate dilution (Example 2; columns 4-12) from the corresponding well of the dilution plate. Each well of columns 4-12 further received 20 μL of 0.020 ng/μL enzyme (Example 3). Accordingly, the final concentration of substrate in each sample well is half of the concentration of the dilution indicated in Table 3 and each well of the assay plate contains a total of 40 μL. Columns 1-3 include product without enzyme in order to produce a standard curve, columns 4-6 include an assay control, and each of columns 7-9 and 10-12, respectively includes an experimental sample. All wells were mixed gently by pipetting. The plate was sealed and incubated in a thermocycler at 37° C. for 20 minutes, after which the enzyme was quenched by addition of 120 μL HPLC-grade acetonitrile to each well. All wells were mixed by pipetting. Sample-to-sample variation was minimized by adding the acetonitrile to the assay plate in the same well order and timing as the order and timing in which the enzyme had been added to the same plate.

After addition of acetonitrile, samples were separately transferred to corresponding wells of a Sirocco protein precipitation filter plate positioned over a clean collection plate. The filter plate was sealed and the filter apparatus was centrifuged for 2 minutes at 2,000×g. The collection plate was visually inspected to ensure that all wells contained filtrate. If liquid remained in the filter following centrifugation, wells may be mixed by pipetting and the filter apparatus subsequently centrifuged a second time to collect additional filtrate.

Filtered reaction mixture was analyzed by ultra-performance liquid chromatography coupled to fluorescence detection (UPLC-FLD). A Waters Acquity I-Class UPLC with fluorescence detector or similarly capable devices may be used in conjunction with a BEH amide column, 1.7 μm, 2.1×100 mm. After turning on the fluorescence detector lamp, the UPLC instrument lines were wet-primed and the pump was started with the initial method conditions of 93% Eluent B (90% acetonitrile, 20 mM ammonium formate, pH 3.5; prepared from 100 mL of 200 mM ammonium formate, pH 3.5 and 900 mL acetonitrile), 0.3 mL/min. Pressure and fluorescence chromatograms were monitored until both were stable, approximately 20 minutes. The sample collection plate was placed in the autosampler unit of the instrument. Sampling was configured for 3 μL injections of sample and a 10 minute run time. Sample analysis proceeded in accordance with the instrument method and processing method identified in Tables 4 and 5, respectively. Data was analyzed to determine the enzyme kinetics according to Example 5.

TABLE 4
Instrument method

| | |
|---|---|
| Mobile phase | A: 10% acetonitrile, 20 mM ammonium |
| | formate, pH 3.5 |
| | B: 90% acetonitrile, 20 mM ammonium |
| | formate, pH 3.5 |
| | Needle wash/seal wash: 90% acetonitrile |
| Flow rate | 0.3 mL/min |
| Gradient | Isocratic, 93% B |
| Column temperature | 40° C. ± 5° C. |
| Sample injection volume | 3 μL |
| Autosampler temperature | 4° C. ± 2.5° C. |
| Fluorescence detection | Ex 308 nm, Em 370 nm |
| Run time | 10 minutes |

TABLE 5
Processing method

| | |
|---|---|
| Integration algorithm | ApexTrac |
| Integration start/end | 1.5/6.0 minutes |
| Minimum area | 50000 μV*sec |
| Peak width | 12.24 sec |
| Liftoff % | 0.0% |
| Touchdown | 0.5% |
| RT window | 5.0% |
| Component info | IdoA-4MU |
| | RT 2.452 minutes |

### Example 5

### Determination of Enzyme Kinetics

The concentration of IdoA-4MU in each well was determined using a product standard curve, and the rate of product formation was plotted against the substrate concentration. The data were fitted to the Michaelis-Menten model to obtain Vₘₐₓ (τM/min) and Kₘ (τM); k_{cat}(s⁻¹) was calculated by dividing Vₘₐₓ with the enzyme concentration [E] (μM).

In particular, the IdoA-4MU product standard curve was produced by first calculating the average peak area for IdoA-4MU product standard concentration. The % CV was calculated for each of the three replicates and recorded. Next, a linear regression curve of the average peak area vs. the IdoA-4MU product standard concentration (μM) was generated using, e.g., an Empower processing method or Excel. The slope, intercept, and R² values were determined and recorded.

Velocities were calculated from the product peak areas. For each injection of assay control and sample at each substrate concentration, the peak area was converted to a concentration using the standard curve parameters and divided by 20 minutes to obtain the velocity (μM/min). The average velocity and % CV of the triplicate determination for each substrate concentration were determined and recorded.

The reportable values k_{cat} and Kₘ were determined by performing non-linear regression utilizing each replicate velocity and the corresponding substrate concentration. Non-linear regression fit was performed using the Michaelis-Menten equation: v₀=Vₘₐₓ[S]/(Kₘ+[S]). Vₘₐₓ was divided by the total enzyme concentration in the reaction and divided by 60 seconds/minute to obtain k_{cat}(s⁻¹). The enzyme concentration used to calculate k_{cat} assumes 100% formylglycine and 100% correctly folded active sites that are catalytically competent. The enzyme concentration in the reaction, used for calculating k_{cat} from Vₘₐₓ, was 1.27×10−4 μM, a value calculated based on a molecular mass of 78.8 kDa (˜g/mol) as determined by MADLI-TOF. The determination of enzyme kinetics may utilize software capable of performing non-linear regression. The k_{cat}(s⁻¹) and Kₘ (μM) of the non-linear regression fit are reported.

### Example 6

### Multi-Step Reaction for Determination of I2S Specific Activity

The present Example provides a multi-step reaction for determination of I2S specific activity. Such an exemplary multi-step reaction is outlined in the schematic of FIG. 5. As is exemplified in FIG. 5, a method as described in the present example includes two steps. In a first step, 4-methylumbelliferyl α-L-idopyranosiduronic acid 2-sulfate (IdoA2S-MU) is hydrolyzed to sulfate and 4-methylumbelliferyl α-L-idopyranosiduronic acid (IdoA-MU). The first step is catalyzed by idursulfase or iduronate-2-sulfatase (I2S). In a second step, 4-methylumbelliferyl α-L-idopyranosiduronic acid (IdoA-MU) is hydrolyzed to idopyranosiduronic acid (IdoA) and 4-methylumbelliferone (4-MU). The second step is catalyzed by iduronidase (IDUA). The schematic further illustrates as part of the second step a high pH quench generating an anionic version of 4-MU that is measurable by fluorescence.

In the present Example, the enzyme activity of iduronate-2-sulfatase (I2S) was measured using a two-step reaction sequence in a 96-well plate format. The plate included assay control samples of enzyme and test samples of enzyme. All samples were diluted (e.g., based on the official A280 concentration to a target concentration of 25 ng/mL) in assay buffer (10 mM acetate pH 4.7 with 0.03 mg/mL BSA) and mixed with 0.5 mM IdoA2S-MU substrate on ice. It is noted that, once diluted, the sample solutions are not stable in the absence of substrate. Therefore dilution should be carried out such that the time of the samples in diluted state without substrate becomes minimal. Also dilutions should be done on ice using ice-cold buffer solutions to prevent the progression of the reaction when mixed with substrate.

The reaction mixtures were next incubated in a thermocycler at 37° C. for 30 minutes in assay buffer (10 mM acetate pH 4.7 with 0.03 mg/mL BSA). The reaction was stopped by adding 0.5 μg/well of IDUA dissolved in McIlvaine's buffer (0.4 M sodium phosphate dibasic, 0.2M sodium citrate, 0.02% sodium azide, pH 4.5), which serves as a stop solution for the first step (see FIG. 5; hydrolysis of IdoA2S-MU to sulfate and IdoA-MU) of the reaction as well.

Following addition of IDUA, the plate of samples was incubated in a thermocycler at 37° C. for an additional 4 hours to complete the second step (see FIG. 5; hydrolysis of IdoA-MU to IdoA and 4-MU) of the reaction. The reaction was stopped by adding carbonate stop solution (0.5 M Sodium carbonate, 0.025% Triton X-100, pH 10.7).

The second step includes a high pH quench. The high pH quench of the second step reaction generates the anionic form of 4-MU, the generation of which was measured by fluorescence using excitation and emission wavelengths of 365 nm and 450 nm, respectively. The amount of 4-MU generated in the enzyme-catalyzed reaction was interpolated from a 4-MU standard curve fitted using a second order polynomial function.

For preparation of a calibration curve, diluent was prepared for calibration samples (for use in producing a standard curve) according to the content of the reaction mixtures. An exemplary diluent for calibration samples may be prepared by combining 1 part assay buffer (10 mM acetate pH 4.7 with 0.03 mg/mL BSA), 1 part McIlvaine's buffer (0.4 M sodium phosphate dibasic, 0.2M sodium citrate, 0.02% sodium azide, pH 4.5), and 5 parts stop solution (0.5 M Sodium carbonate, 0.025% Triton X-100, pH 10.7). A 0.1 mM solution of 4-MU was prepared by adding 20 μL of 10 mM 4-MU into 1980 μL of the calibration diluent. 4-MU solution was diluted into diluent in rows 1-3 of a 96-well plate (having rows 1 to 12 and columns A to H) to produce a dilution sequence, each well having a total volume of 400 μL solution, and each row including the following concentrations of 4-MU (from col. A to col. H): 1.4 μM, 1.2 μM, 1 μM, 0.8 μM, 0.6 μM, 0.4 μM, 0.2 μM, and 0 μM.

To prepare sample analysis plates, 200 μL each of the 36 calibration mixtures described above are transferred to a new 96-well plate. An equal volume of sample reaction is placed into each of the remaining wells. Samples are typically included in triplicate. Accordingly, in an exemplary dilution series, carried out in a 96-well plate, reportable values for up to 23 samples can be obtained using this test method.

To measure sample and calibration fluorescent signals, analysis plates were read for fluorescence signal with excitation and emission wavelengths of 365 and 450 nm, respectively, with an auto-cutoff wavelength of 435 nm.

Enzyme activity may be recorded in U/mL for in-process samples, where unit [U] is defined as the amount of enzyme required to release 1 μmol of sulfate per minute. In certain instances, enzyme activity can be reported in U/mg, e.g., for mock Drug Substance (DS), development DS, DS, and and Drug Product (DP) samples, including stability samples by dividing the activity by the concentration of enzyme added to the 1^{st} step reaction according to the following equation:

$$Specific activity [ \frac{µmol}{min * mg} - U \text{/} mg ] = \frac{Activity}{{C}_{pr}}$$

Where, Cₚᵣ=protein concentration of the sample in mg/L

### Example 7

Use of Multi-Step Reaction for Determination of I2S Specific Activity for in-Process Sampling

The assay of the Example 6 was used for the purpose of in-process sampling. In-process samples were diluted to recommended dilution level(s) depending on the sample type or protein concentration. The reportable value for in-process sample types is expressed in U/mL, where, for the purposes of the present Example, one U is defined as the quantity of I2S required to hydrolyze one micromole of sulfate per minute. This test method provides the U/mL value for the tested in-process samples. Specific activity can be determined for in-process samples by dividing the U/mL value by the official protein concentration (mg/mL) obtained by either titer ELISA or concentration by A280 using an extinction coefficient.

### Example 8

Use of Multi-Step Reaction for Determination of I2S Specific Activity for DS and/or DP Sampling

The assay of the Example 6 was used for the purpose of DS and DP sampling. Drug Substance (DS) and Drug Product (DP) sample types were diluted to defined target concentrations for use in the first step reaction. The dilution required was calculated based on the official protein concentration by A280 using an extinction coefficient (e.g., SoloVPE A280). DS/DP samples should be diluted based on the official A280 concentration to a target concentration of 25 ng/mL. The reportable value for DS and DP samples types is expressed in U/mg, where one U is defined as the quantity of IDS required to hydrolyze one micromole of sulfate per minute.

### Other Embodiments

While we have described a number of embodiments of this invention, it is apparent that our basic disclosure and examples may be altered to provide other embodiments that utilize the compounds and methods of this invention. Therefore, it will be appreciated that the scope of this invention is to be defined by the appended claims rather than by the specific embodiments that have been represented by way of example. All references cited herein are hereby incorporated by reference.

What is claimed is:

1. A method of determining the potency of iduronate-2-sulfatase (I2S), comprising the steps of:
contacting a sample comprising iduronate-2-sulfatase (I2S) with a substrate containing a terminal iduronate-2-sulfate under conditions that permit the I2S-catalyzed desulfation of the substrate, wherein the I2S-catalyzed desulfation of the substrate is associated with a detectable signal; and
detecting the detectable signal, thereby determining one or more kinetic parameters or the specific activity of the I2S,
wherein the one or more kinetic parameters or the specific activity is indicative of the potency and active properties of I2S.

2. The method of claim 1, wherein the substrate is defined by a structure of formula I: 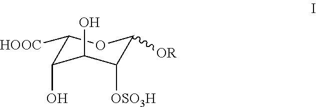
or a suitable salt thereof wherein R is hydrogen, a carbohydrate domain optionally substituted with a detectable moiety, an oxygen protecting group, a detectable moiety, or an optionally substituted group selected from the group consisting of C₁₋₁₂ aliphatic, phenyl, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, 5- to 6-membered heteroaryl having 1-4 heteroatoms selected from oxygen, nitrogen, or sulfur, 7- to 10-membered saturated or partially unsaturated bicyclic carbocyclyl, 7- to 10-membered saturated or partially unsaturated bicyclic heterocyclyl having 1-4 heteroatoms selected from oxygen, nitrogen, or sulfur, 7- to 10-membered bicyclic heteroaryl having 1-4 heteroatoms selected from oxygen, nitrogen, or sulfur, or 8- to 10-membered bicyclic aryl; and [symbol] indicates an α- or β-anomer, or a mixture thereof.

3. The method of claim 2, wherein the detectable group is a fluorescent group.

4. The method of claim 2, wherein the detectable group is detectable via chemiluminescence or ultraviolet/visible absorbance spectroscopy.

5. The method of claim 3, wherein R is 4-methylumbelliferyl (4MU).

6. The method of any one of the preceding claims, wherein the substrate is 4-methylumbelliferyl-α-L-idopyranosiduronic acid 2-sulfate (IdoA2S-4MU):

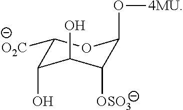

7. The method of any one of the preceding claims, wherein the I2S-catalyzed desulfation generates an I2S product, and the product is IdoA-4MU:

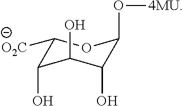

8. The method of any one of the preceding claims, wherein the step of detecting the detectable signal comprises performing chromatography.

9. The method of claim 8, wherein the chromatography is selected from the group consisting of ion chromatography, high-performance liquid chromatography (HPLC), ultra performance liquid chromatography, and combination thereof.

10. The method of claim 8, wherein the step of detecting the detectable signal comprises performing ultra-performance or high-performance liquid chromatography coupled to fluorescence detection.

11. The method of any one of claims 8-10, wherein the chromatography includes at least one column selected from a BEH amide, HILIC, RP, or CSH column.

12. The method of any one of the preceding claims, wherein the step of detecting the detectable signal comprises determining the amount of the product as compared to a control.

13. The method of claim 12, wherein the control is a pre-determined amount of the product.

14. The method of claim 12, wherein the control is a product standard curve.

15. The method of any one of the preceding claims, wherein the step of detecting the detectable signal comprises determining the rate of product formation.

16. The method of any one of the preceding claims, the one or more kinetic parameters are selected from the group consisting of Vₘₐₓ, Kₘ, k_{cat}, specific activity, and combination thereof.

17. The method of claim 16, wherein the I2S-catalyzed desulfation generates an I2S product, and the one or more kinetic parameters are determined by fitting data obtained from analyzing the product formation to the Michaelis-Menten model or other kinetic models suitable to determine kinetic parameters.

18. The method of any one of the preceding claims, wherein the sample is a drug substance, a drug product, or a stability sample of drug substance and drug product.

19. The method of any one of the preceding claims, wherein the conditions that permit I2S-catalyzed desulfation of the substrate comprise incubation at about 37° C. for about 20 minutes, a buffer pH of approximately 4-5, and in the presence of BSA.

20. The method of any one of the preceding claims, wherein the conditions that permit the I2S-catalyzed desulfation of the substrate comprise between 0 and 0.4 mg/mL BSA.

21. The method of any one of the preceding claims, wherein the method further comprises a step of quenching the desulfation by addition of acetonitrile, or another organic solvent, water-miscible or not miscible, or by using heat denaturation.

22. The method of claim 1 or 2, wherein the method further comprises contacting the sample with iduronidase (IDUA) under conditions that permit generation of detectable 4-MU.

23. The method of claim 22, wherein the contacting of the sample with IDUA is after the I2S-catalyzed desulfation of the substrate.

24. The method of claim 22 or 23, wherein the conditions that permit generation of detectable 4-MU include adding a high pH quench reagent.
